(19) United States
(12) Patent Application Publication
(10) Pub. No.: US 2008/0280987 A1
(43) Pub. Date: Nov. 13, 2008

(54) Methods of inhibiting angiogenesis and treating angiogenesis-associated diseases

(76) Inventors: Neil P. Desai, Los Angeles, CA (US); Vuong Trieu, Calabasas, CA (US)

Correspondence Address: MORRISON & FOERSTER LLP, 755 PAGE MILL RD, PALO ALTO, CA, 94304-1018, US

(21) Appl. No.: 11/897,724

(22) Filed: Aug. 31, 2007

Related U.S. Application Data
(60) Provisional application No. 60/841,719, filed on Aug. 31, 2006.

(51) Int. Cl. A61K 31/165; A61P 35/00; A61P 27/02; A61P 9/00; A61P 17/06

(52) U.S. Cl. 514/616

(57) ABSTRACT

The invention provides methods of inhibiting angiogenesis in an individual by administering a composition (such as protein containing composition) comprising colchicine or thiocolchicine dimer. The composition is in an amount that is effective in inhibiting angiogenesis but is in some embodiments insufficient to induce significant cytotoxicity in the individual. The methods described herein are useful for treating angiogenesis-associated diseases, such as age-related macular degeneration, diabetic retinopathy, rheumatic arthritis, psoriasis, and cancer.

### RELATED APPLICATIONS

This application claims priority benefit of provisional patent application No. 60/841,719, filed on Aug. 31, 2006, the disclosure of which is herein incorporated by reference in its entirety.

### TECHNICAL FIELD

This application pertains to methods of inhibiting angiogenesis and treating angiogenesis-associated diseases. Specifically, the application pertains to methods of inhibiting angiogenesis and treating angiogenesis-associated diseases by administering an effective amount of a composition comprising a colchicine or thiocolchicine dimer.

### BACKGROUND

Angiogenesis is a highly regulated biological process by which new blood vessels are formed. Uncontrolled angiogenesis leads to many diseases. One such disease is age-related macular degeneration (“AMD”), which is characterized by the invasion of new blood vessels into different structures of the eye such as macular and retinal pigment epithelium. Another disease in which angiogenesis is implicated is rheumatoid arthritis, where blood vessels in the synovial lining of the joints undergo angiogenesis. In addition to forming new vascular networks, the endothelial cells release factors and reactive oxygen species that lead to pannus growth and cartilage destruction. Uncontrolled angiogenesis is also associated with diseases such as diabetic retinopathy, psoriasis, restenosis, and neovascular glaucoma.

In addition, angiogenesis is also involved in tumor formation and metastasis. It has been shown, for example, that tumors which enlarge to greater than about two millimeters in diameter must obtain their own blood supply and do so by inducing the growth of new capillary blood vessels. After these new blood vessels become embedded in the tumor, they provide nutrients and growth factors essential for tumor growth and facilitate metastasis of tumor cells.

Anti-angiogenic agents that specifically target angiogenesis have been developed for treating angiogenesis-associated diseases. See, e.g., U.S. Pat. No. 6,919,309; U.S. Pat. App. Pub. No. 2006/0009412; and PCT App. Pub. Nos. WO04/027027 and WO05/117876. In addition, agents that target established vasculatures (so called “Vascular Targeting Agents” or VTAs) have also been developed. These agents are believed to function by selectively destabilizing the microtubule cytoskeleton of endothelial cells, causing a profound alteration in the shape of the cells which ultimately leads to occlusion of the blood vessel and shutdown of blood flow. See, e.g., WO 2005/113532.

Thiocolchicine dimers are hydrophobic compounds that have been previously described. See, e.g., U.S. Pat. No. 6,627,774. These compounds have dual mechanisms of action, i.e., the compounds have both anti-microtubule activities and topoisomerase I inhibitory activities. Raspaglio et al., Biochem. Pharmacol. 2005, 69(1): 113-21. Nanoparticle albumin-bound formulations of thiocolchicine dimers Nab-5404 and Nab-5676 have been developed as cytotoxic chemotherapeutic agents for treating cancer. See, for example, Bernacki et al., Proc. Amer. Assoc. Cancer Res., vol. 46, 2005 #2390 and PCT Pat. App. No. PCT/US2006/006167. It was found that Nab-5404, when administered intravenously at 24 mg/kg, qd×5, was capable of inducing complete tumor regressions and cures in A121 ovarian tumor xenograft.

The disclosures of all publications, patents, patent applications and published patent applications referred to herein are hereby incorporated herein by reference in their entireties.

### BRIEF SUMMARY OF THE INVENTION

The invention in one aspect provides a method of inhibiting angiogenesis in an individual, comprising administering to the individual a composition comprising a colchicine or thiocolchicine dimer (such as IDN5404), wherein the composition is in an amount that is effective in inhibiting angiogenesis, and wherein the amount of the composition is insufficient to induce significant cytotoxicity in the individual. In some embodiments, there is provided a method of inhibiting angiogenesis in an individual, comprising administering to the individual an effective amount of a composition comprising a colchicine or thiocolchicine dimer (such as IDN5404), wherein the amount of the colchicine or thiocolchicine dimer (such as IDN5404) in the composition (i.e., the amount per administration) is less than about any of 2, 3, 4, 5, 6, 7, 8, 9, 10, 11, 12, 13, 14, or 15 mg/m² body surface. In some embodiments, there is provided a method of inhibiting angiogenesis in an individual, comprising administering to the individual an effective amount of a composition comprising a colchicine or thiocolchicine dimer (such as IDN5404), wherein the amount of the colchicine or thiocolchicine dimer (such as IDN5404) in the composition is less than about any of 0.05, 0.08, 0.1, 0.2, 0.3, 0.4, 0.5, 0.6, 0.7, 0.8, 0.9, 1, 2, 3, 4, 5, 6, 7, 8, 9, or 10 mg/kg. In some embodiments, the composition is administered at least about any of once every three weeks, once every two weeks, once a week, twice a week, three times a week, four times a week, five times a week, six times a week, or daily. In some embodiments, the composition is administered (with or without breaks) for at least about any of 1, 2, 3, 4, 5, 6, 7, 8, 9, 10, 11, 12, or more month(s). In some embodiments, the composition is administered via any of intravenous, intraocular, intraarterial, oral, topical, or inhalational routes. In some embodiments, the individual has cancer. In some embodiments, the individual has non-tumorous angiogenesis-associated disease.

The compositions used in methods described herein may further comprise a biocompatible polymer, such as a carrier protein. For example, in some embodiments, there is provided a method of inhibiting angiogenesis in an individual, comprising administering to the individual a composition comprising a carrier protein (such as albumin) and a colchicine or thiocolchicine dimer (such as IDN5404), wherein the composition is in an amount that is effective in inhibiting angiogenesis, and wherein the amount of the composition is insufficient to induce significant cytotoxicity in the individual. In some embodiments, there is provided a method of inhibiting angiogenesis in an individual, comprising administering to the individual an effective amount of a composition comprising a carrier protein (such as albumin) and a colchicine or thiocolchicine dimer (such as IDN5404), wherein the amount of the colchicine or thiocolchicine dimer (such as IDN5404) in the composition is less than about any of 2, 3, 4, 5, 6, 7, 8, 9, 10, 11, 12, 13, 14, or 15 mg/m² body surface. In some embodiments, there is provided a method of inhibiting angiogenesis in an individual, comprising administering to the individual an effective amount of a composition comprising a carrier protein (such as albumin) and a colchicine or thiocolchicine dimer (such as IDN5404), wherein the amount of the colchicine or thiocolchicine dimer (such as IDN5404) in the composition is less than about any of 0.05, 0.08, 0.1, 0.2, 0.3, 0.4, 0.5, 0.6, 0.7, 0.8, 0.9, 1, 2, 3, 4, 5, 6, 7, 8, 9, or 10 mg/kg. In some embodiments, the carrier protein-containing composition is substantially free (such as free) of surfactant. In some embodiments, the composition is administered at least about any of once every three weeks, once every two weeks, once a week, twice a week, three times a week, four times a week, five times a week, six times a week, or daily. In some embodiments, the composition is administered (with or without breaks) for at least about any of 1, 2, 3, 4, 5, 6, 7, 8, 9, 10, 11, 12, or more month(s). In some embodiments, the composition is administered via any of intravenous, intraocular, intraarterial, oral, topical, or inhalational routes. In some embodiments, the individual has cancer. In some embodiments, the individual has non-tumorous angiogenesis-associated disease.

The compositions used in methods described herein may comprise particles (such as microparticles or nanoparticles) comprising a carrier protein (such as albumin) and a colchicine or thiocolchine dimer. For example, in some embodiments, there is provided a method of inhibiting angiogenesis in an individual, comprising administering to the individual a composition comprising nanoparticles comprising a carrier protein (such as albumin) and a colchicine or thiocolchine dimer (such as IDN5404), wherein the composition is in an amount that is effective in inhibiting angiogenesis, and wherein the amount of the composition is insufficient to induce significant cytotoxicity in the individual. In some embodiments, there is provided a method of inhibiting angiogenesis in an individual, comprising administering to the individual an effective amount of a composition comprising nanoparticles comprising a carrier protein (such as albumin) and a colchicine or thiocolchicine dimer (such as IDN5404), wherein the amount of the colchicine or thiocolchicine dimer (such as IDN5404) in the composition is less than about any of 2, 3, 4, 5, 6, 7, 8, 9, 10, 11, 12, 13, 14, or 15 mg/m² body surface. In some embodiments, there is provided a method of inhibiting angiogenesis in an individual, comprising administering to the individual an effective amount of a composition comprising nanoparticles comprising a carrier protein (such as albumin) and a colchicine or thiocolchicine dimer (such as IDN5404), wherein the amount of the colchicine or thiocolchicine dimer (such as IDN5404) in the composition is less than about any of 0.05, 0.08, 0.1, 0.2, 0.3, 0.4, 0.5, 0.6, 0.7, 0.8, 0.9, 1, 2, 3, 4, 5, 6, 7, 8, 9, or 10 mg/kg. In some embodiments, the composition is administered at least about any of once every three weeks, once every two weeks, once a week, twice a week, three times a week, four times a week, five times a week, six times a week, or daily. In some embodiments, the composition is administered (with or without breaks) for at least about any of 1, 2, 3, 4, 5, 6, 7, 8, 9, 10, 11, 12, or more month(s). In some embodiments, the composition is administered via any of intravenous, intraocular, intraarterial, oral, topical, or inhalational routes. In some embodiments, the individual has cancer. In some embodiments, the individual has non-tumorous angiogenesis-associated disease.

In some embodiments, the particles in the composition have an average diameter of no greater than about 200 nm. In some embodiments, the particle-containing composition is substantially free (such as free) of surfactant. In some embodiments, the weight ratio of the protein (such as albumin) to the colchicine or thiocolchicine dimer in the composition is about 18:1 or less, such as about 9:1 or less. In some embodiments, the colchicine or thiocolchicine dimer is coated with the carrier protein (such as albumin). In some embodiments, the particles in the composition have an average diameter of no greater than about 200 nm and the composition is substantially free (such as free) of surfactant. In some embodiments, the particles (particularly nanoparticles) in the composition have an average diameter of no greater than about 200 nm and the colchicine or thiocolchine dimer is coated with protein (such as albumin). Other combinations of the above characteristics are also contemplated. In some embodiments, the particle composition is Nab-5404. In some embodiments, the particle composition is Nab-5676. Particle compositions comprising other colchicine or thiocolchicine dimers may also comprise one or more of the above characteristics.

The colchicine or thiocolchicine dimers described herein comprise two (same or different) subunits of colchicine, thiocolchicine, or derivatives thereof. In some embodiments, the colchicine or thiocolchicine dimer comprises at least one thiocolchicine subunit. In some embodiments, the colchicine or thiocolchicine dimer comprises two thiocolchicine subunits. In some embodiments, the colchicine or thiocolchicine dimer is a compound of formula (I):

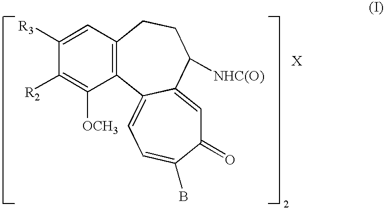

wherein the B in each subunit is either a methoxy or a methylthio group, R₂ is methoxy, hydroxyl, or methylenedioxy when taken together with R₃, R₃ is methoxy, hydroxyl, or methylenedioxy when taken together with R₂, and X is a linking group. In some embodiments, X contains at least one carbon atom.

In some embodiments, the colchicine or thiocolchicine dimer has is a compound of formula (II):

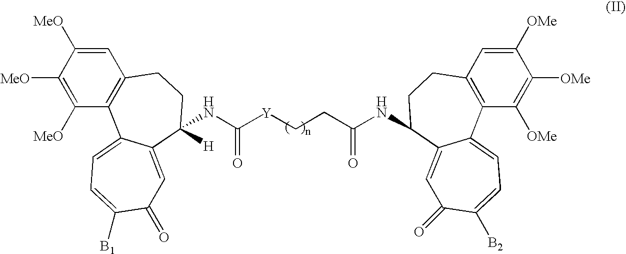

wherein B₁ is a methoxy or a methylthio group, B₂ is a methoxy or a methylthio group, n is an integer from 0 to 8, Y is a CH₂ group or, when n is 1, can also be a group of formula NH. In some embodiments, n is any of (such as selected from the group consisting of) 0, 1, 2, 3, 4, 5, 6, 7, or 8. In some embodiments, n is 1. In some embodiments, n is 1 and Y is NH. In some embodiments, n is 2.

In some embodiments, both B₁ and B₂ are methoxy groups. In some embodiments, both B₁ and B₂ are methylthio groups. In some embodiments, B₁ is a methoxy group and B₂ is a methylthio group. In some embodiments, B₁ is a methylthio group and B₂ is a methoxy group. In some embodiments, the colchicine or thiocolchicine dimer is any of (and in some embodiments selected from the group consisting of): IDN5404, IDN5676, IDN5800, and IDN5801. In some embodiments, the colchicine or thiocolchicine dimer is IDN5404. In some embodiments, the colchicine or thiocolchicine dimer is IDN5676.

In some embodiments, there is provided a method of inhibiting angiogenesis in an individual, comprising administering to the individual a composition comprising nanoparticles comprising albumin and any of IDN5404, IDN5676, IDN5800, and IDN5801 (hereinafter designated as “Nab-5404,” “Nab-5676,” “Nab-5800” and “Nab-5801,” respectively). In some embodiments, there is provided a method of inhibiting angiogenesis in an individual, comprising administering to the individual Nab-5404 (or any of Nab-5676, Nab-5800, and Nab-5801), wherein the Nab-5404 (or any of Nab-5676, Nab-5800, and Nab-5801) is in an amount that is effective in inhibiting angiogenesis, and wherein the amount of Nab-5404 (or any of Nab-5676, Nab-5800, and Nab-5801) is insufficient to induce significant cytotoxicity in the individual. In some embodiments, there is provided a method of inhibiting angiogenesis in an individual, comprising administering to the individual an effective amount of Nab-5404 (or any of Nab-5676, Nab-5800, and Nab-5801), wherein the amount of Nab-5404 (or any of Nab-5676, Nab-5800, and Nab-5801) is less than about any of 2, 3, 4, 5, 6, 7, 8, 9, 10, 11, 12, 13, 14, or 15 mg/m² body surface. In some embodiments, there is provided a method of inhibiting angiogenesis in an individual, comprising administering to the individual an effective amount of Nab-5404 (or any of Nab-5676, Nab-5800, and Nab-5801), wherein the amount of Nab-5404 (or any of Nab-5676, Nab-5800, and Nab-5801) is less than about any of 0.05, 0.08, 0.1, 0.2, 0.3, 0.4, 0.5, 0.6, 0.7, 0.8, 0.9, 1, 2, 3, 4, 5, 6, 7, 8, 9, or 10 mg/kg. In some embodiments, the composition is administered at least about any of once every three weeks, once every two weeks, once a week, twice a week, three times a week, four times a week, five times a week, six times a week, or daily. In some embodiments, the composition is administered (with or without breaks) for at least about any of 1, 2, 3, 4, 5, 6, 7, 8, 9, 10, 11, 12, or more month(s). In some embodiments, the composition is administered via any of intravenous, intraocular, intraarterial, oral, topical, or inhalational routes. In some embodiments, the individual has cancer. In some embodiments, the individual has non-tumorous angiogenesis-associated disease.

Methods described herein are generally useful for treatment of angiogenesis-associated diseases. In some embodiments, the angiogenesis-associated disease is a non-tumorous angiogenesis-associated disease, including for example eye diseases (such as macular degeneration, diabetic retinopathy, or neovascular glaucoma), cardiovascular diseases (such as restenosis and atherosclerosis), skin diseases (such as psoriasis), and arthritis (such as rheumatic arthritis).

For example, in some embodiments, there is provided a method of treating a non-tumorous angiogenesis-associated disease in an individual, comprising administering to the individual a composition comprising a colchicine or thiocolchicine dimer, wherein the composition is in an amount that is effective in treating the non-tumorous angiogenesis-associated disease. In some embodiments, there is provided a method of inhibiting angiogenesis in an individual having non-tumorous angiogenesis-associated disease, comprising administering to the individual a composition comprising a colchicine or thiocolchicine dimer, wherein the composition is in an amount that is effective in inhibiting angiogenesis in the individual. In some embodiments, the amount of the composition is insufficient to induce significant cytotoxicity in the individual. In some embodiments, the individual does not have cancer. In some embodiments, the individual does not have a tumor-related disease.

In some embodiments, there is provided a method of treating a non-tumorous angiogenesis-associated disease in an individual, comprising administering to the individual a composition comprising a carrier protein (such as albumin) and a colchicine or thiocolchicine dimer (such as IDN5404), wherein the composition is in an amount that is effective in treating the non-tumorous angiogenesis-associated disease in an individual. In some embodiments, there is provided a method of inhibiting angiogenesis in an individual having non-tumorous angiogenesis-associated disease, comprising administering to the individual a composition comprising a carrier protein (such as albumin) and a colchicine or thiocolchicine dimer (such as IDN5404), wherein the composition is in an amount that is effective in inhibiting angiogenesis in the individual. In some embodiments, the amount of the composition is insufficient to induce significant cytotoxicity in the individual. In some embodiments, the individual does not have cancer. In some embodiments, the individual does not have a tumor-related disease.

In some embodiments, there is provided a method of treating a non-tumorous angiogenesis-associated disease in an individual, comprising administering to the individual a composition comprising particles (such as nanoparticles) comprising a carrier protein (such as albumin) and a colchicine or thiocolchine dimer (such as IDN5404), wherein the composition is in an amount that is effective in treating the non-tumorous angiogenesis-associated disease. In some embodiments, there is provided a method of inhibiting angiogenesis in an individual having non-tumorous angiogenesis-associated disease, comprising administering to the individual a composition comprising particles (such as nanoparticles) comprising a carrier protein (such as albumin) and a colchicine or thiocolchine dimer (such as IDN5404), wherein the composition is in an amount that is effective in inhibiting angiogenesis in the individual. In some embodiments, the amount of the composition is insufficient to induce significant cytotoxicity in the individual. In some embodiments, the individual does not have cancer. In some embodiments, the individual does not have a tumor-related disease.

In some embodiments, there is provided a method of treating a non-tumorous angiogenesis-associated disease in an individual, comprising administering to the individual Nab-5404 (or Nab-5676, Nab-5800, or Nab-5801), wherein the Nab-5404 (or Nab-5676, Nab-5800, or Nab-5801) is in an amount that is effective in treating the non-tumorous angiogenesis-associated disease. In some embodiments, there is provided a method of inhibiting angiogenesis in an individual having non-tumorous angiogenesis-associated disease, comprising administering to the individual Nab-5404 (or Nab-5676, Nab-5800, or Nab-5801), wherein the Nab-5404 (or Nab-5676, Nab-5800, or Nab-5801) is in an amount that is effective in inhibiting angiogenesis in the individual. In some embodiments, the amount of Nab-5404 (or Nab-5676, Nab-5800, or Nab-5801) is insufficient to induce significant cytotoxicity in the individual. In some embodiments, the individual has cancer. In some embodiments, the individual has non-tumorous angiogenesis-associated disease.

In some embodiments, the angiogenesis-associated disease is a tumor-related disease, including for example cancer and benign tumor. Cancers that can be treated by methods described herein include, but are not limited to, breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, gliomas, glioblastomas, neuroblastomas, and multiple myeloma. In some embodiments, the cancer is a solid tumor (such as metastatic solid tumor). By way of example, methods of treating cancer are further described below.

For example, in some embodiments, there is provided a method of treating cancer in an individual, comprising administering to the individual a composition comprising a colchicine or thiocolchicine dimer, wherein the composition is in an amount that is effective in treating cancer, and wherein the amount of the composition is insufficient to induce significant cytotoxicity in the individual. In some embodiments, there is provided a method of inhibiting angiogenesis in an individual having cancer, comprising administering to the individual a composition comprising a colchicine or thiocolchicine dimer, wherein the composition is in an amount that is effective in inhibiting angiogenesis, and wherein the amount of the composition is insufficient to induce significant cytotoxicity in the individual.

In some embodiments, there is provided a method of treating cancer in an individual, comprising administering to the individual a composition comprising a colchicine or thiocolchicine dimer, wherein the composition is in an amount that is effective in treating cancer, and wherein the amount of the colchicine or thiocolchicine dimer in the composition is less than about any of 1%, 2%, 3%, 4%, 5%, 6%, 7%, 8%, 9%, 11%, 12%, 13%, 14%, 15%, 20%, 25%, 30%, 40%, or 50% of the corresponding maximum tolerated dose (“MTD”) of the colchicine or thiocolchicine dimer. In some embodiments, there is provided a method of inhibiting angiogenesis in an individual having cancer, comprising administering to the individual a composition comprising a colchicine or thiocolchicine dimer, wherein the composition is in an amount that is effective in inhibiting angiogenesis, and wherein the amount of the colchicine or thiocolchicine dimer in the composition is less than about any of 1%, 2%, 3%, 4%, 5%, 6%, 7%, 8%, 9%, 11%, 12%, 13%, 14%, 15%, 20%, 25%, 30%, 40%, or 50% of the corresponding maximum tolerated dose (“MTD”) of the colchicine or thiocolchicine dimer.

In some embodiments, there is provided a method of treating cancer in an individual (or method of inhibiting angiogenesis in an individual having cancer), comprising administering to the individual a composition comprising a carrier protein (such as albumin) and a colchicine or thiocolchicine dimer (such as IDN5404), wherein the composition is in an amount that is effective in treating cancer (or inhibiting angiogenesis), and wherein the amount of the composition is insufficient to induce significant cytotoxicity in the individual. In some embodiments, there is provided a method of treating cancer in an individual (or inhibiting angiogenesis in an individual having cancer), comprising administering to the individual a composition comprising a carrier protein (such as albumin) and a colchicine or thiocolchicine dimer (such as IDN5404), wherein the composition is in an amount that is effective in treating cancer (or inhibiting angiogenesis), and wherein the amount of the colchicine or thiocolchicine dimer in the composition is less than about any of 1%, 2%, 3%, 4%, 5%, 6%, 7%, 8%, 9%, 11%, 12%, 13%, 14%, 15%, 20%, 25%, 30%, 40%, or 50% of the corresponding MTD of the colchicine or thiocolchicine dimer. In some embodiments, there is provided a method of treating cancer in an individual (or inhibiting angiogenesis in an individual having cancer), comprising administering to the individual a composition comprising particles (such as nanoparticles) comprising a carrier protein (such as albumin) and a colchicine or thiocolchine dimer (such as IDN5404), wherein the composition is in an amount that is effective in treating cancer (or inhibiting angiogenesis), and wherein the amount of the composition is insufficient to induce significant cytotoxicity in the individual. In some embodiments, there is provided a method of treating cancer in an individual (or inhibiting angiogenesis in an individual having cancer), comprising administering to the individual a composition comprising particles (such as nanoparticles) comprising a carrier protein (such as albumin) and a colchicine or thiocolchine dimer (such as IDN5404), wherein the composition is in an amount that is effective in treating cancer (or inhibiting angiogenesis), and wherein the amount of the colchicine or thiocolchicine dimer in the composition is less than about any of 1%, 2%, 3%, 4%, 5%, 6%, 7%, 8%, 9%, 11%, 12%, 13%, 14%, 15%, 20%, 25%, 30%, 40%, or 50% of the corresponding MTD of the colchicine or thiocolchicine dimer. In some embodiments, there is provided a method of treating cancer in an individual (or inhibiting angiogenesis in an individual having cancer), comprising administering to the individual Nab-5404 (or Nab-5676, Nab-5800, or Nab-5801), wherein the Nab-5404 (or Nab-5676, Nab-5800, or Nab-5801) is in an amount that is effective in treating cancer (or inhibiting angiogenesis), and wherein the amount of Nab-5404 (or Nab-5676, Nab-5800, or Nab-5801) is insufficient to induce significant cytotoxicity in the individual. In some embodiments, there is provided a method of treating cancer in an individual (or inhibiting angiogenesis in an individual having cancer), comprising administering to the individual Nab-5404 (or Nab-5676, Nab-5800, or Nab-5801), wherein the Nab-5404 (or Nab-5676, Nab-5800, or Nab-5801) is in an amount that is effective in treating cancer (or inhibiting angiogenesis), and wherein the amount of Nab-5404 (or Nab-5676, Nab-5800, or Nab-5801) is less than about any of 1%, 2%, 3%, 4%, 5%, 6%, 7%, 8%, 9%, 11%, 12%, 13%, 14%, 15%, 20%, 25%, 30%, 40%, or 50% of the corresponding MTD of Nab-5404 (or Nab-5676, Nab-5800, or Nab-5801).

Also provided are unit dosage forms used for methods described herein. In some embodiments, there is provided a unit dosage form comprising a composition comprising colchicine or thiocolchicine dimer in an amount that is effective in inhibiting angiogenesis, wherein the amount of the composition is insufficient to induce significant cytotoxicity in the individual. In some embodiments, there is provided a unit dosage form comprising a composition comprising colchicine or thiocolchicine dimer in an amount that is effective in treating cancer, wherein the amount of colchicine or thiocolchicine dimer in the composition per administration is less than about any of 1%, 2%, 3%, 4%, 5%, 6%, 7%, 8%, 9%, 10%, 11%, 12%, 13%, 14%, 15%, 20%, 25%, 30%, 40%, or 50% of the corresponding MTD for the colchicine or thiocolchicine dimer composition. In some embodiments, the unit dosage form comprises a colchicine or thiocolchicine dimer in an amount that is sufficient to delivery about 0.05 mg/m² to about 100 mg/m² (including for example about 1 mg/m² to about 15 mg/m²) colchicine or thiocolchicine dimer to a subject. In some embodiments, the unit dosage form comprises about 0.05 mg to about 200 mg (including for example about 1 mg to about 30 mg) colchicine or thiocolchicine dimer. In some embodiments, the unit dosage form further comprises a carrier protein, such as carrier proteins described herein. In some embodiments, the unit dosage form comprises nanoparticles comprising a colchicine or thiocolchicine dimer and a carrier protein, such as nanoparticle compositions described herein.

Also provided herein are pharmaceutical compositions suitable for methods described above. The pharmaceutical compositions may comprise a colchicine or thiocolchicine dimer and a pharmaceutically acceptable carrier suitable for delivery to the eye (such as intraocular injection or topical application to the eye) and/or intraarterial injection. The pharmaceutical compositions may further comprise carrier proteins, such as carrier proteins described herein. For example, in some embodiments, the pharmaceutical composition comprises a colchicine or thiocolchicine dimer, a carrier protein, and a pharmaceutically acceptable carrier suitable for delivery to the eye (such as intraocular injection or topical application to the eye). In some embodiments, the pharmaceutical composition comprises a colchicine or thiocolchicine dimer, a carrier protein, and a pharmaceutically acceptable carrier suitable for intraarterial injection. The pharmaceutical compositions may comprise particles (such as microparticles or nanoparticles) comprising a carrier protein and a colchicine or thiocolchicine dimer, as described herein.

Further described herein are kits and articles of manufacture that are useful for methods described herein.

It is to be understood that one, some, or all of the properties of the various embodiments described herein may be combined to form other embodiments of the present invention.

### BRIEF DESCRIPTION OF THE FIGURES

FIG. 1A shows the effect of Nab-5404 on microtubule formation using MX-1 breast carcinoma cells. FIG. 1B shows the effect of Nab-5676 on microtubule formation using MX-1 breast carcinoma cells.

FIGS. 2A-2C show micrographs of stained microtubule network in MX-1 cells after treatment with control vehicle (PBS, 2A), 0.6 μg/ml Nab-5404 (2B), and μg/ml Nab-5676 (2C).

FIG. 3 (FIGS. 3A-3D) shows the anti-angiogenenic activity of Nab-5404, Nab-5676 and CA4P as evaluated by microvessel/tubule formation. Cells were incubated with various compounds on day 1 and stained on day 12. FIGS. 3A-3D provide micrographs of tubule formation after treatment with control vehicle (3C), 0.01 μg/ml Nab-5404 (3A), 0.01 μg/ml Nab-5676 (3B), and 0.01 μg/ml CA4P (3D).

FIG. 4 shows a comparison of the tubule length for cells treated with Nab-5404, Nab-5676, and CA4-P across a concentration range of 0.0 to 100 μg/ml.

FIG. 5 (FIGS. 5A-5D) shows the anti-angiogenenic activity of Nab-5404 and Nab-5676 as evaluated by microvessel/tubule formation or disruption. Cells were incubated with various compounds on day 8 and stained on day 11. FIGS. 5A-5C provide micrographs of tubule formation after treatment with control vehicle (5A), 0.01 g/ml Nab-5404 (5B), and 0.01 μg/ml Nab-5676 (5C). FIG. 5D shows a comparison of the tubule length for cells treated with Nab-5404 and Nab-5676 across a concentration range of 0.0 to 10 μg/ml.

FIG. 6 (FIGS. 6A-6F) shows the anti-angiogenenic activity of Nab-5404 as evaluated by microvessel/tubule formation or disruption. Cells were incubated with various concentrations of the compound on day 11 and stained on day 12. FIGS. 6A to 6F provide micrographs of tubule formation and disruption after treatment with control vehicle (6A), 0.001 μg/ml Nab-5404 (6B), 0.01 μg/ml Nab-5404 (6C), 0.1 μg/ml Nab-5404 (6D), 1 μg/ml Nab-5404 (6E), and 10.0 μg/ml Nab-5404 (6F).

FIG. 7 (FIGS. 7A-7F) shows the anti-angiogenenic activity of Nab-5676 as evaluated by microvessel/tubule formation or disruption. Cells were incubated with various concentrations of the compound on day 11 and stained on day 12. FIGS. 7A to 7F show micrographs of tubule formation and disruption after treatment with control vehicle (7A), 0.001 μg/ml Nab-5676 (7B), 0.01 μg/ml Nab-5676 (7C), 0.1 μg/ml Nab-5676 (7D), 1 μg/ml Nab-5676 (7E), and 10.0 μg/ml Nab-5676 (7F).

FIG. 8 (FIGS. 8A-8C) shows the anti-angiogenenic activity of CA4P as evaluated by microvessel/tubule formation or disruption. Cells were incubated with various concentrations of the compound on day 11 and stained on day 12. FIGS. 8A to 8C show micrographs of tubule formation and disruption after treatment with 0.01, 0.1, and 1.0 μg/ml CA4P, respectively.

FIG. 9 shows a comparison of the anti-angiogenenic activities of Nab-5404, Nab-5676 and CA4P as evaluated by microvessel or tubule formation or disruption. Cells were incubated with compounds on day 11 and stained on day 12. The figure shows the tubule length for cells treated with each compound across a concentration range of 0.0 to 10 μg/ml.

FIG. 10 (FIGS. 10A-10B) shows the effects of Nab-5404 on HT-29 tumor growth in a xenograft murine model using a 2 cycle, low-dose/high dose schedule. First cycle doses were 1.7, 2.5 or 3.4 mg/kg, days 0-14; second cycle doses were 20, 30, or 40 mg/kg, days 15-30. FIG. 10A shows the mean tumor volume (n=10) from day 0 to day 40. FIG. 10B shows the mean percent weight loss of the mice over days 0 to 40.

FIG. 11 (FIGS. 11A-11B) shows the effects of Nab-5676 on HT-29 tumor growth in a xenograft murine model using a 2 cycle, low-dose/high dose schedule. First cycle doses were 1.7, 2.5 or 3.4 mg/kg, days 0-14; second cycle doses were 20, 30, or 40 mg/kg, days 15-30. FIG. 11A shows the mean tumor volume (n=10) from day 0 to day 40. FIG. 11B shows the mean percent weight loss of the mice over days 0 to 40.

FIG. 12 (FIGS. 12A-12B) shows the effects of CA4P on HT-29 tumor growth in a xenograft murine model. HT-29 tumors were treated with 100 mg/kg after they had reached a volume of 900 mm3. FIG. 12A shows the mean tumor volume (n=10) from day 28 to day 41; squares=vehicle control, diamonds=CA4P treatment. FIG. 12B shows the mean percent weight loss of the mice over days 28 to 41; squares=vehicle control, diamonds=CA4P treatment.

### DETAILED DESCRIPTION OF THE INVENTION

The present invention is based, in part, on our observation that compositions comprising a thiocolchicine dimer, specifically, albumin-containing nanoparticle formulations of thiocolchicine dimer, more specifically, albumin-containing nanoparticle formulations of IDN-5404 (“Nab-5404”) and albumin-containing nanoparticle formulations of IDN-5676 (“Nab-5676”), are effective in inhibiting microvessel formation and disrupt established microvessels in vitro. Trieu et al., 97^{th} AACR Annual Meeting, Abstract No. 3823. The IC50 values for these activities are significantly lower than those required for in vitro cytotoxicity activities of the compositions. We further observed that compositions comprising a thiocolchicine dimer, specifically, albumin-containing nanoparticle formulations of thiocolchicine dimer (such as Nab-5404 and Nab-5676), are effective in preventing tumor growth in vivo at a dose that is significantly lower than the corresponding maximum tolerated dose (MTD) of the compositions. These observations suggest that compositions comprising a thiocolchicine dimer or an analog thereof (such as a colchicine dimer) possess anti-angiogenic and vascular targeting activities that are independent of their cytotoxic effects. When administered at an effective and non-cytotoxic amount, the compositions are capable of selectively targeting growth of new blood vessels and blocking blood flow without causing significant cell death in the target tissue.

An effective and noncytotoxic amount of colchicine or thiocolchicine dimer (i.e., an amount that is insufficient to induce significant cytotoxicity) is desirable for treating non-tumorous angiogenesis-associated diseases in order to minimize the cytotoxic effects of the compound. An effective and noncytotoxic amount is also advantageous for treating cancer. Traditional chemotherapy with a cytotoxic agent is typically carried out at a dose that is either the same or close to the maximum tolerated dose of the agent in order to maximize the cytotoxic effect of the agent. This high-dose schedule, however, requires an extended treatment-free period to allow recovery of normal host cells. In the meantime, tumor cells may also resume growth during the treatment free period. This could increase the risk of the developing drug resistant tumor cells. A noncytotoxic dose (i.e., amount) of a colchicine or thiocolchicine dimer allows treatment without significant breaks in the treatment cycle, thus reduces the risk of developing drug resistance. Furthermore, a noncytotoxic dose minimizes the possibility of developing apparent systemic toxicity (such as weight loss) and side effects induced by the drug.

Accordingly, the invention in one aspect provides a method of inhibiting angiogenesis (including targeting established vascularization) in an individual, comprising administering to the individual a composition comprising a colchicine or thiocolchicine dimer, wherein the composition is in an amount that is effective in inhibiting angiogenesis, and wherein the amount of the composition is insufficient to induce significant cytotoxicity in the individual. In some embodiments, the method comprises administering to the individual a composition comprising a carrier protein (such as albumin) and a colchicine or thiocolchicine dimer, wherein the composition is in an amount that is effective in inhibiting angiogenesis, and wherein the amount of the composition is insufficient to induce significant cytotoxicity in the individual.

In another aspect, the invention provides a method of treating a non-tumorous angiogenesis-associated disease in an individual (or inhibiting angiogenesis in an individual having a non-tumorous angiogenesis-associated disease), comprising administering to the individual a composition comprising a colchicine or thiocolchicine dimer, wherein the composition is in an amount that is effective in treating the non-tumorous angiogenesis-associated disease (or inhibiting angiogenesis). In some embodiments, the method comprises administering to the individual a composition comprising a carrier protein and a colchicine or thiocolchicine dimer, wherein the composition is in an amount that is effective in treating the non-tumorous angiogenesis-associated disease (or inhibiting angiogenesis).

In another aspect, the invention provides a method of treating a tumor-related disease (such as cancer) in an individual, comprising administering to the individual a composition comprising a colchicine or thiocolchicine dimer, wherein the composition is in an amount that is effective in treating the tumor-related disease, and wherein the amount of the composition is insufficient to induce significant cytotoxicity in the individual. In some embodiments, the method comprises administering to the individual a composition comprising a carrier protein and a colchicine or thiocolchicine dimer, wherein the composition is in an amount that is effective in treating cancer, and wherein the amount of the composition is insufficient to induce significant cytotoxicity in the individual.

Also provided herein are unit dosage forms comprising a composition comprising colchicine or thiocolchicine dimer in an amount that is effective in inhibiting angiogenesis, wherein the amount of the composition in the unit dosage form is insufficient to induce significant cytotoxicity.

Also provided herein are pharmaceutical compositions comprising a colchicine or thiocolchicine dimer, optionally a carrier protein, and a pharmaceutically acceptable carrier suitable for delivery to the eye (such as intraocular injection and topical application to the eye) and/or intraarterial injection.

Also provided are kits and articles of manufacture that are useful for methods described herein.

General reference to “the composition” or “compositions” includes and is applicable to compositions of the invention. The invention also provides pharmaceutical compositions comprising the components described herein.

The term “individual” is a mammal, including humans. An individual includes, but is not limited to, human, bovine, equine, feline, canine, rodent, or primate. In some embodiments, the individual is human. In some embodiments, the individual is an experimental animal model for studying angiogenesis or angiogenesis-associated disease(s).

As is understood by one of skill in the art, reference to “about” a value or parameter herein includes (and describes) embodiments that are directed to that value or parameter per se. For example, description referring to “about X” includes description of “X”.

The disclosure includes all stereoisomers of the compounds referred to herein, including enantiomers and diastereomers. Unless stereochemistry is explicitly indicated in a structure, the disclosed structure is intended to embrace all possible stereochemical variants. The disclosure includes all enantiomers of any chiral compound disclosed, in either substantially pure levorotatory or dextrorotatory form, or in a racemic mixture, or in any ratio of enantiomers. The disclosure includes any diastereomers of the compounds referred to in the above formulas in substantially pure diastereomeric form and in the form of mixtures in all ratios. The disclosure also includes all solvates of the compounds referred to herein, including all hydrates of the compounds referred to herein. The disclosure also includes all polymorphs, including crystalline and non-crystalline forms of the compounds referred to herein. The disclosure also includes all salts of the compounds referred to herein, particularly pharmaceutically-acceptable salts. Metabolites and prodrugs of the compounds disclosed herein are also embraced by the disclosure. In all uses of the compounds disclosed herein, the disclosure also includes use of any or all of the stereochemical, enantiomeric, diastereomeric, solvates, hydrates, polymorphic, crystalline, non-crystalline, salt, pharmaceutically acceptable salt, metabolite and prodrug variations of the compounds as described.

It is understood that aspects and embodiments of the invention described herein include “consisting” and/or “consisting essentially of” aspects and embodiments.

### Methods of the Inhibiting Angiogenesis

The invention in one aspect provides a method of inhibiting angiogenesis in an individual by administering a composition (such as a carrier protein-containing composition) comprising colchicine or thiocolchicine dimer. The composition is in an amount that is effective in inhibiting angiogenesis. The amount of the composition administered is, however, insufficient to induce significant cytotoxicity in the individual.

“Angiogenesis” used herein refers to the process of generating new blood vessels in a tissue or organ. Angiogenesis typically begins with the erosion of the basement membrane of a blood vessel by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new vessel.

“Inhibiting angiogenesis” refers to reducing, impeding, or suppressing angiogenesis in one or more tissues in an individual, including for example eye tissue, cardiovascular tissue, skin tissue, arthritic tissue, and tumor tissue. Inhibition of angiogenesis can be achieved by affecting one or more steps of the angiogenesis process, for example, by attenuating the migration and survival of activated endothelial cells, preventing “sprouting” of cells from parent vessel, and/or preventing formation of new blood vessels. Changes in microvessel density are also encompassed within the term “inhibiting angiogenesis.” The term “inhibiting angiogenesis” also encompasses disrupting established vasculature. “Disrupting established vasculature” refers to the ability to occlude, dissolve, or otherwise affect existing vasculature formed by angiogenesis. The disruption of the vasculature can be reversible or irreversible, partial or complete.

Accordingly, methods provided herein encompass one or more of the following aspects: inhibiting (such as reducing, impeding, or preventing) endothelial cell migration, inhibiting (such as reducing, impeding, or preventing) “sprouting” of endothelial cells from parent vessel, inhibiting (such as reducing, impeding, or preventing) formation of new blood vessels, and targeting (such as occluding, disrupting, or destroying) established vasculature formed by angiogenesis. In some embodiments, there is provided a method of inhibiting (such as reducing, impeding, or preventing) blood vessel formation in an individual. In some embodiments, there is provided a method of disrupting (such as occluding, dissolving, or otherwise affecting) established vasculature. In some embodiments, there is provided a method of reducing density of microvessels in a tissue of an individual.

“Effective amount” refers to an amount or dose of the composition which, upon single or multiple dose administration to the individual, provides a desired effect in the individual. For example, the composition is “in an amount that is effective in inhibiting angiogenesis” if the amount of the composition is sufficient to reduce, impede, or prevent angiogenesis (such as one or more aspects of angiogenesis) in one or more tissues in an individual. The term “effective amount” used in the treatment context refers to an amount of a compound or composition sufficient to treat a specified disorder, condition or disease such as ameliorate, palliate, lessen, and/or delay one or more of its symptoms. An effective amount can be determined in vitro and/or vivo. Methods of determining an effective amount of a composition for inhibiting angiogenesis are known in the art.

In some embodiments, the amount of the composition is effective in inhibiting angiogenesis in an eye tissue. In some embodiments, the amount of the composition is effective in inhibiting angiogenesis in a cardiovascular tissue. In some embodiments, the amount of the composition is effective in inhibiting angiogenesis in a skin tissue. In some embodiments, the amount of the composition is effective in inhibiting angiogenesis in an arthritic tissue. In some embodiments, the amount of the composition is effective in inhibiting angiogenesis in a tumor tissue. In some embodiments, the amount of the composition is effective in inhibiting new vessel formation. In some embodiments, the amount of the composition is effective in inhibiting “sprouting” of endothelial cells from parent blood vessel. In some embodiments, the amount of the composition is effective in inhibiting migration of endothelial cells. In some embodiments, the amount of the composition is effective to disrupt established vasculature. In some embodiments, the amount of the composition is effective in inhibiting angiogenesis (such as any one or more aspects of angiogenesis) by at least about any of 5%, 10%, 20%, 30%, 40%, 50%, 60%, 70%, 80%, 90%, or more.

A composition is “in an amount that is insufficient to induce significant cytotoxicity” (also referred to as “noncytotoxic amount”) if the amount of the composition is insufficient to allow the colchicine or thiocolchicine dimer to cause significant cell death in an individual. Cytotoxicity can be measured by one or more of the following. For example, noncytoxic amount can be determined based on an in vitro cell viability assay. The noncytotoxic amount can be an amount that is insufficient to cause about 50% or more cell death in an in vitro cell viability assay. In some embodiments, the amount of the composition is insufficient to cause about any of 40% or more, 30% or more, 20% or more, 10% or more, 5% or more, 4% or more, 3% or more, 2% or more, or 1% or more cell death in an in vitro cell viability assay. In some embodiments, the amount of the composition is insufficient to cause any measurable cell death in an in vitro cell viability assay. Suitable cells for in vitro viability assays include, but are not limited to, tumor cells (such as MX-1 breast carcinoma cell lines, HepG2 hepatoma cell lines, and HT-29 colon carcinoma cell lines) and normal cells (such as primary rat hepatocytes). Noncytotoxic amount can also be determined based on in vivo assay of drug toxicity. For example, the noncytotoxic amount can be an amount that is insufficient to kill about 50% or more of the test population in in vivo cytotoxicity assays. In some embodiments, the amount of the colchicine or thiocolchicine dimer is insufficient to kill about any of 40% or more, 30% or more, 20% or more, 10% or more, 5% or more, 4% or more, 3% or more, 2% or more, or 1% or more test population in an in vivo cytotoxicity assay. In some embodiments, the amount of the composition is insufficient to cause any death in a test population in an in vivo drug toxicity assay. Noncytotoxic amount can also be determined based on the amount of colchicine or thiocolchicine dimer that is required to induce apparent systemic toxicity (such as weight loss) in an individual, that is, the amount of the drug is noncytotoxic if it does not induce any apparent systemic toxicity. For example, in some embodiments, a noncytotoxic amount is an amount that induces less than about 15% (including for example less than about any of 10%, 8%, 5%, or less) of weight loss.

In some embodiments, the colchicine and thiocolchincine dimer is in an amount that does not induce drug resistance over an extended administration period (such as 6 months or more), with or without breaks. In some embodiments, the colchicine and thiocolchicine dimer is in an amount that does not induce significant side effects in the individual (such as side effects that typically accompany chemotherapy). Side effects that typically accompany chemotherapy include, for example, dehydration, diarrhea, nausea, vomiting, vision loss or disturbance, and anemia. In some embodiments, the colchicine or thiocolchicine dimer is in an amount that does not cause hematological toxicity (such as myelosuppression).

In some embodiments, the amount of the colchicine or thiocolchicine dimer in the composition (e.g., at each administration) is less than about any of 1, 2, 3, 4, 5, 6, 7, 8, 9, 10, 11, 12, 13, 14, 15, 20, 30, 40, 50, 60, 70, 80, 90, or 100 mg/m². For example, the amount of the colchicine or thiocolchicine dimer can range from about 0.25 mg/m² body surface to about 100 mg/m², about 0.25 mg/m² to about 50 mg/m², 0.25 mg/m² body surface to about 15 mg/m², about 0.25 mg/m² to about 10 mg/m², about 0.25 mg/m² to about 8 mg/m², about 0.25 mg/m² to about 4 mg/m², and about 0.25 mg/m² to about 2 mg/m². In some embodiments, the amount of colchicine or thiocolchcine dimer composition per administration is any of about 0.25 mg/m² to about 0.5 mg/m², about 0.5 mg/m² to about 1 mg/m², about 1 mg/m² to about 2 mg/m², about 2 mg/m² to about 3 mg/m², about 3 mg/m² to about 4 mg/m², about 4 mg/m² to about 5 mg/m², about 5 mg/m² to about 6 mg/m², about 6 mg/m² to about 7 mg/m², about 7 mg/m² to about 8 mg/m², about 8 mg/m² to about 9 mg/m², about 9 mg/m² to about 10 mg/m², about 10 mg/m² to about 15 mg/m², about 15 mg/m² to about 20 mg/m², about 20 mg/m² to about 50 mg/m², about 50 mg/m² to about 100 mg/m².

In some embodiments, the amount of the colchicine or thiocolchicine dimer in the composition (e.g., at each administration) is less than about any of 0.01, 0.05, 0.08, 0.1, 0.2, 0.3, 0.4, 0.5, 0.6, 0.7, 0.8, 0.9, 1, 2, 3, 4, 5, 6, 7, 8, 9, or 10 mg/kg. For example, the amount of the colchicine or thiocolchicine dimer can range from about 0.05 mg/kg to about 10 mg/kg, about 0.05 mg/kg to about 5 mg/kg, about 0.05 mg/kg to about 1 mg/kg, about 0.05 mg/kg to about 0.5 mg/kg, about 0.05 mg/kg to about 0.3 mg/kg, about 0.05 mg/kg to about 0.2 mg/mg, or about 0.05 mg/mg to about 0.1 mg/kg. In some embodiments, the amount of colchicine or thiocolchcine dimer composition per administration is any of about 0.01 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 0.6 mg/kg, about 0.6 mg/kg to about 0.7 mg/kg, about 0.7 mg/kg to about 0.8 mg/kg, about 0.8 mg/kg to about 0.9 mg/kg, about 0.9 mg/kg to about 1 mg/kg, about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 6 mg/kg, about 6 mg/kg to about 7 mg/kg, about 7 mg/kg to about 8 mg/kg, about 8 mg/kg to about 9 mg/kg, or about 9 mg/kg to about 10 mg/kg.

In some embodiments, the amount of a colchicine or thiocolchcine dimer in the composition is included in any of the following ranges: about 0.05 mg to about 0.1 mg, about 0.1 mg to about 0.2 mg, about 0.2 mg to about 0.3 mg, about 0.3 mg to about 0.4 mg, about 0.4 mg to about 0.5 mg, about 0.5 mg to about 1 mg, about 1 mg to about 5 mg, about 5 to about 10 mg, about 10 mg to about 15 mg, about 15 mg to about 20 mg, about 20 mg to about 25 mg, about 25 mg to about 50 mg, about 50 mg to about 100 mg, or about 100 to about 200 mg.

Dosing frequency for the compositions includes, but is not limited to, at least about any of once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day(s). In some embodiments, the interval between each administration is constant. For example, the administration can be carried out daily, every two days, every three days, every four days, every five days, every six days, weekly, four out of five weeks, three out of four weeks, or two out of three weeks. In some embodiments, the administration can be carried out twice daily, three times daily, or more frequent.

The administration of the composition can be extended over an extended period of time, such as from about a month up to about three years. For example, the dosing regime can be extended over a period of any of about 1, 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The composition described herein can be administered to an individual via any route known in the art, including, but not limited to, intravenous, intraperitoneal, intraocular, intra-arterial, intrapulmonary, oral, intravesicular, intramuscular, intra-tracheal, subcutaneous, intraocular, intrathecal, transdermal, transpleural, intraarterial, topical, inhalational (e.g., as mists of sprays), transmucosal (such as via nasal mucosa), subcutaneous, transdermal, gastrointestinal, intraarticular, intracisternal, intraventricular, rectal (i.e., via suppository), vaginal (i.e., via pessary), intracranial, intraurethral, intrahepatic, and intratumoral. In some embodiments, the composition is administered systemically. In some embodiments, the composition is administered locally. In some embodiments, the composition is administered via any of intravenous, intraocular, intraarterial, oral, topical, or inhalational routes. In some embodiments, the composition is administered intravenously. In some embodiments, the composition is administered intraocularly. In some embodiments, the composition is administered intraarterially. In some embodiments, the composition is administered topically.

When the method is directed to inhibition of angiogenesis in an eye tissue, the composition can be administered directly to the eye or the eye tissue. The composition can be administered topically to the eye, as in eye drops. The composition can also be administered via injection to the eye or to the tissues associated with the eye. The composition can be administered via intraocular injection, periocular injection, subretinal injection, intravetreal injection, trans-septal injection, subscleral injection, intrachoroidal injection, intracameral injection, subconjunctival injection, sub-Tenon's injection, retrobulbar injection, peribulbar injection, or posterior juxtascleral delivery. The composition may be administered, for example, to the vitreous, aqueous humor, sclera, conjunctiva, the area between the sclera and conjunctiva, the retina choroids tissues, macula, or other area in or proximate to the eye of an individual. For a description of exemplary periocular routes for retinal drug delivery, see Periocular routes for retinal drug delivery, Raghava et al. (2004), Expert Opin. Drug Deliv. 1(1):99-114, which is incorporated herein in its entirety. The composition can also be administered to the individual as an implant. Preferred implants are biocompatible and/or biodegradable sustained release formulations which gradually release the compounds over a period of time. Ocular implants for drug delivery are well-known in the art. See, e.g., U.S. Pat. Nos. 5,501,856, 5,476,511, and 6,331,313. The composition can also be administered to the individual using iontophoresis, including, but not limited to, the iontophoretic methods described in U.S. Pat. No. 4,454,151 and U.S. Pat. App. Pub. Nos. 2003/0181531 and 2004/0058313.

### Methods of Treating Angiogenesis-Associated Diseases

The methods described herein are generally useful for treatment of angiogenesis-associated diseases. “Angiogenesis-associated disease” refers to a disease or disorder where angiogenesis is one aspect of the disease. The angiogenesis-associated disease may be caused by abnormal angiogenesis. In some embodiments, the angiogenesis-associated disease is mediated at least in part by angiogenesis. In some embodiments, angiogenesis is necessary for the development of the angiogenesis-associated disease. Angiogenesis-associated diseases are known in the art, and include, for example, non-tumorous angiogenesis-associated diseases such as macular degeneration, diabetic retinopathy, rheumatoid arthritis, and other diseases described herein. In some embodiments, the angiogenesis-associated disease is a tumor-related disease, such as cancer or benign tumor.

As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, stabilized (i.e., not worsening) state of disease, preventing or delaying spread of disease, preventing or delaying occurrence or recurrence of disease, delay or slowing of disease progression, amelioration of the disease state, and remission (whether partial or total). Also encompassed by “treatment” is a reduction of pathological consequence of an angiogenesis-associated disease. The methods of the invention contemplate any one or more of these aspects of treatment.

### Treatment of Non-Tumorous Angiogenesis-Associated Diseases

The methods described herein are useful for the treatment of non-tumorous angiogenesis-associated diseases in an individual. In some embodiments, the individual does not have cancer. In some embodiments, the individual does not have a tumor-related disease.

In some embodiments, the methods are useful for treatment of non-tumorous angiogenesis-associated diseases in the eye tissue, such as in the cornea, retina, macula, and choroids. The methods are generally useful for preventing blindness, loss of vision (such as loss of visual acuity or visual field), and/or other consequences resulting from a variety of eye diseases. In some embodiments, there is provided a method for treating macular degeneration, including age-related macular degeneration (AMD). AMD is clinically characterized by progressive loss of central vision which occurs as a result of damage to the photoreceptor cells in an area of the retina called the macula. AMD has been broadly classified into two clinical states: a wet form and a dry form, with the dry form making up to 80-90% of total cases. The dry form is characterized clinically by the presence of macular drusen, which are localized deposits between the retinal pigment epithelium (RPE) and the Brunch's membrane, and by geographic atrophy characterized by RPE cell death with overlying photoreceptor atrophy. Wet AMD, which accounts for approximately 90% of serious vision loss, is associated with neovascularization in the area of the macular and leakage of these new vessels. The accumulation of blood and fluid can cause retina detachment followed by rapid photoreceptor degeneration and loss of vision. It is generally accepted that the wet form of AMD is preceded by and arises from the dry form.

The methods provided herein are particularly useful in the treatment or inhibition of the wet form of macular degeneration. Inhibition of angiogenesis in the eye tissue also prevents or delays the transition from the dry form of macular degeneration to the wet form of macular degeneration. The invention thus also provides methods for the treatment of the dry form of macular degeneration. The invention also encompasses methods of treating or preventing one or more aspects or symptoms of macular degeneration, including, but not limited to, loss of photoreceptor cells, loss of vision (including for example visual acuity and visual field), and retina detachment. Other related aspects, such as photoreceptor degeneration, RPE degeneration, retinal degeneration, chorioretinal degeneration, cone degeneration, retinal dysfunction, retinal damage, damage to the Brunch's membrane, loss of RPE function, loss of integrity of the histoarchitecture of the cells and/or extracellular matrix of the normal macular, loss of function of the cells in the macular, and photoreceptor dystrophy, are also encompassed.

Other non-tumorous angiogenesis-associated eye diseases that can be treated by methods described herein include, but are not limited to, neovascularization of the retina, neovascularization of the cornea (such as that caused by trachoma, infections, inflammation, transplantations or trauma), diabetic retinopathy, diabetic retinal edema, diabetic macula edema, ischemic retinopathy, hypertensive retinopathy, occlusive retinopathy, retinopathy of prematurity, neovascularization subsequent to trauma, neovascularization subsequent to infection, neovascularization subsequent to transplantation, neovascularization subsequent to retinal detachment or retinal degeneration, neovascular glaucoma, anterior chamber and/or anterior chamber angle neovascularization, choroidal neovascularization (CNV), subretinal neovascularization, retrolental fibroplasias, ocular histoplasmosis syndrome, myopic degeneration, angioid streaks, uveitis, rubeosis, retrolental fibroplasias, ocular histoplasmosis, and idiopathic central serous chorioretinopathy. In some embodiments, the eye disease is diabetic retinopathy. In some embodiments, the eye disease is neovascular glaucoma.

In some embodiments, there are provided methods of treating vascular diseases, including cardiovascular diseases such as atherosclerosis, restenosis, atheroma, and haemangioma. Atherosclerosis is a form of chronic vascular injury in which some of the normal vascular smooth cells (VSMC) in the artery wall change their nature and develop dense networks of capillaries in atherosclerotic plaques. These fragile microvessels can cause hemorrhages, leading to blood clotting, with a subsequent decreased blood flow to the heart muscle and heart attack. Restenosis typically occurs after coronary artery bypass surgery, endarterectomy, and heart transplantation, and particularly after heart balloon angioplasty, atherectomy, laser ablation or endovascular stenting. It involves extensive growth of microvessels. By inhibiting angiogenesis in the cardiovascular tissue, the methods provided herein are useful for treating these cardiovascular diseases.

In some embodiments, the methods are useful for treatment of rheumatoid arthritis, where blood vessels in the joints undergo angiogenesis to form an extensively vascularized tissue that invades and destroys the cartilage. By inhibiting angiogenesis in the arthritic tissue, the methods provided herein are useful for treating rheumatoid arthritis. Methods of treating hemophiliac joints are also provided.

In some embodiments, the methods are useful for treating angiogenesis-associated skin diseases, including, but are not limited to, psoriasis, scleroderma, neovascularization as a consequence of infection (e.g., cat scratch disease, bacterial ulceration, etc.), and other skin disorders. In some embodiments, the methods are useful for treating psoriasis. Psoriasis is a chronic skin disease occurring in approximately 3% of the population worldwide. Histologic studies, including electron microscopy, have established that alterations in the blood vessel formation of the skin are a prominent feature of psoriasis. Inhibition of angiogenesis in the skin tissue is thus useful for treating psoriasis.

Other angiogenesis-associated diseases that can be treated by methods of the present invention include, but are not limited to, Osler-Webber Syndrome, hereditary hemorrhagic telangiectasia, plaque neovascularization, telangiectasia, angiofibroma, angioma, wound granularization, endometriosis, and the like. In addition, the invention is useful for treatment of nasal polyps, especially in cystic fibrosis patients.

Besides treating angiogenesis-associated diseases, methods described herein can also be used to modulate or prevent the occurrence of normal physiological conditions associated with angiogenesis. For example, the inventive method can be used to attenuate neovascularization associated with ovulation, implantation of an embryo, placenta formation, etc, and are thus useful for birth control purposes.

Provided herein are methods of treating various diseases described herein. In some embodiments, there is provided a method of treating a non-tumorous angiogenesis-associated disease in an individual, comprising administering to the individual a composition (such as a composition comprising a carrier protein) comprising a colchicine or thiocolchicine dimer, wherein the composition is in an amount that is effective in treating the non-tumorous angiogenesis-associated disease. In some embodiments, there is provided a method of treating an angiogenesis-associated eye disease (including for example macular degeneration, diabetic retinopathy, or neovascular glaucoma) in an individual, comprising administering to the individual a composition (such as a composition comprising a carrier protein) comprising a colchicine or thiocolchicine dimer, wherein the composition is in an amount that is effective in treating the eye disease. In some embodiments, there is provided a method of treating a cardiovascular disease (such as restenosis and atherosclerosis), comprising administering to the individual a composition (such as a composition comprising a carrier protein) comprising a colchicine or thiocolchicine dimer, wherein the composition is in an amount that is effective in treating the cardiovascular disease. In some embodiments, there is provided a method of treating an angiogenesis-associated skin disease (such as psoriasis), comprising administering to the individual a composition (such as a composition comprising a carrier protein) comprising a colchicine or thiocolchicine dimer, wherein the composition is in an amount that is effective in treating the skin disease. In some embodiments, there is provided a method of treating arthritis (such as rheumatoid arthritis), comprising administering to the individual a composition (such as a composition comprising a carrier protein) comprising a colchicine or thiocolchicine dimer, wherein the composition is in an amount that is effective in treating arthritis.

In some embodiments, the colchicine or thiocolchine dimer composition is administered at least about any of once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day(s). In some embodiments, the interval between each administration is constant. For example, the administration can be carried out daily, every two days, every three days, every four days, every five days, every six days, weekly, four out of five weeks, three out of four weeks, or two out of three weeks. In some embodiments, the administration can be carried out twice daily, three times daily, or more frequent. The administration of the composition can be extended (with or without breaks) over an extended period of time, such as from about a month up to about three years. For example, the dosing regime can be extended (with or without breaks) over a period of any of about 1, 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

### Methods of Treating Tumor-Related Diseases by Inhibiting Angiogenesis

The methods described herein are also useful for inhibition of angiogenesis in tumor tissues and for treating tumor-related diseases such as cancers and benign tumors.

Inhibiting angiogenesis (for example by inhibiting new blood vessel formation or by targeting established blood vessels) prevents sufficient nutrients and oxygen from being supplied to the tumor to support growth beyond a given size. Because angiogenesis is involved in both primary tumor growth and metastasis, the methods provided herein are capable of inhibiting neoplastic growth of tumor at the primary site as well as preventing metastasis of tumors at the secondary sites. In some embodiments, the colchicine or thiocolchicine dimer inhibits new blood vessel formation. In some embodiments, the colchicine or thiocolchicine dimer composition disrupts established tumor vasculature. For example, the compositions may be effective in the selective occlusion, dissolution, or otherwise affect, whether reversible or irreversible, partial or complete, of tumor vasculature (sometimes proliferating tumor vasculature).

The invention provides methods of treating tumor-related disease by administering an effective amount of a composition comprising a colchicine or thiocolchicine dimer, wherein the amount of the composition is insufficient to induce significant cytotoxicity (also referred to as a “noncytotoxic dose”). Traditional chemotherapy with a cytotoxic agent is typically carried out at a dose that is either the same or close to the maximum tolerated dose of the agent in order to maximize the cytotoxic effect of the agent. This high-dose schedule, however, requires an extended treatment-free period to allow recovery of normal host cells. In the meantime, tumor cells may also resume growth during the treatment free period. This could increase the risk of the developing drug resistant tumor cells. A noncytotoxic dose of a colchicine or thiocolchicine dimer allows cancer treatment without significant breaks in the treatment cycle, thus reduces the risk of developing drug resistance.

To provide an example, methods of treating cancer are further described herein. It is understood that this description generally applies to all tumor-related diseases, including benign tumor.

In some embodiments, there is provided a method of treating cancer in an individual, comprising administering to the individual a composition comprising a colchicine or thiocolchicine dimer, wherein the composition is in an amount that is effective in treating cancer, and wherein the amount of colchicine or thiocolchicine dimer in the composition per administration is less than about any of 1%, 2%, 3%, 4%, 5%, 6%, 7%, 8%, 9%, 10%, 11%, 12%, 13%, 14%, 15%, 20%, 25%, 30%, 40%, or 50% of the corresponding MTD. “Corresponding MTD” used herein refers to the MTD of the same (or similar) colchicine or thiocolchicine dimer in a same (or similar) formulation following a same or similar dosing schedule or a traditional dosing schedule. In some embodiments, the amount of colchicine or thiocolchicine dimer in the composition per administration is between about 1% to about 50% of the corresponding MTD, including for example any of about 1% to about 40%, about 1% to about 30%, about 1% to about 25%, about 1% to about 20%, about 1% to about 15%, about 1% to about 12%, about 1% to about 10%, about 1% to about 8%, about 1% to about 5%, about 1% to about 3%, of the corresponding MTD. The MTD for a colchicine or thiocolchicine dimer is known or can be easily determined by a person skilled in the art. For example, the MTD for Nab-5404 following a weekly schedule is about 90-100 mg/m² body surface.

In some embodiments, the colchicine or thiocolchine dimer composition is administered at least about any of once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day(s). In some embodiments, the interval between each administration is constant. For example, the administration can be carried out daily, every two days, every three days, every four days, every five days, every six days, weekly, four out of five weeks, three out of four weeks, or two out of three weeks. In some embodiments, the administration can be carried out twice daily, three times daily, or more frequent. The administration of the composition can be extended (with or without breaks) over an extended period of time, such as from about a month up to about three years. For example, the dosing regime can be extended (with or without breaks) over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

Cancers that can be treated by methods of the present invention include, but are not limited to, carcinoma, lymphoma, blastoma, sarcoma, and leukemia. More particular examples of such cancers include, but are not limited to, squamous cell cancer, lung cancer (including small cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, heptoma, breast cancer, colon cancer, endometrical or uterine carcinoma, salivary gland carcinoma, kidney or renal cancer, prostate cancer, vulval cancer, thyroid cancer, hepatic carcinoma, head and neck cancer, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome. The methods described herein are particularly useful for treating cancers that involve extensive angiogenesis, such as squamous cell carcinoma.

In some embodiments, there are provided methods of treating primary tumors. In some embodiments, there are provided methods of treating metastatic cancer (that is, cancer that has metastasized from the primary tumor). In some embodiments, there are provided methods of treating cancer at advanced stage(s). In some embodiments, there are provided methods of treating breast cancer (which may be HER2 positive or HER2 negative), including, for example, advanced breast cancer, stage IV breast cancer, locally advanced breast cancer, and metastatic breast cancer. In some embodiments, the cancer is lung cancer, including, for example, non-small cell lung cancer (NSCLC, such as advanced NSCLC), small cell lung cancer (SCLC, such as advanced SCLC), and advanced solid tumor malignancy in the lung. In some embodiments, the cancer is ovarian cancer, head and neck cancer, gastric malignancies, melanoma (including metastatic melanoma), colorectal cancer, pancreatic cancer, and solid tumors (such as advanced solid tumors). In some embodiments, the cancer is any of (and in some embodiments selected from the group consisting of) breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, gliomas, glioblastomas, neuroblastomas, and multiple myeloma. In some embodiments, the cancer is a solid tumor.

The methods described herein may be practiced in an adjuvant setting. “Adjuvant setting” refers to a clinical setting in which an individual has had a history of cancer, and generally (but not necessarily) been responsive to therapy, which includes, but is not limited to, surgery (such as surgical resection), radiotherapy, and chemotherapy. However, because of their history of the cancer, these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated. The methods provided herein may also be practiced in a neoadjuvant setting, i.e., the method may be carried out before the primary/definitive therapy. In some embodiments, the individual has previously been treated. In some embodiments, the individual has not previously been treated. In some embodiments, the treatment is a first line therapy.

The methods described herein may be carried out in conjunction with other methods of cancer treatment (for example, in a combination therapy setting). For example, in some embodiments, the method comprises a first therapy comprising administering a composition comprising a colchicine or thiocolchicine dimer, and a second therapy. In some embodiments, the second therapy is chemotherapy. In some embodiments, the second therapy is radiation therapy. In some embodiments, the second therapy is surgery. The first and second therapy can be carrier out either simultaneously or sequentially (i.e., the first therapy is carrier out before or after the second therapy). In some embodiments, the method comprises co-administering (including simultaneously or sequentially) a composition comprising a colchicine or thiocolchicine dimer with a second chemotherapeutic agent to an individual, wherein the composition is effective in inhibiting angiogenesis in the individual, and wherein the composition is in an amount that is insufficient to induce cytotoxicity in the individual. In some embodiments, the second chemotherapeutic agent is a cytotoxic agent.

### Composition Comprising Colchicine or Thiocolchicine Dimers

### Colchicine or Thiocolchicine Dimers

The methods described herein comprise administration of compositions comprising a colchicine or thiocolchicine dimer. “Colchicine or thiocolchicine dimer” used herein refers to a compound containing two (same or different) subunits of colchicine, thiocolchicine, or derivatives thereof. “Derivatives” of colchicine or thiocolchicine include, but are not limited to, compounds that are structurally similar to colchicine or thiocolchine or are in the same general chemical class as colchicine and thiocolchicine. Generally, the derivative or analog of colchicine or thiocolchine retains similar biological, pharmacological, chemical and/or physical properties (including, for example, functionality) of colchicine or thiocolchicine. In some embodiments, the colchicine or thiocolchicine dimer comprises at least one thiocolchicine subunit. In some embodiments, the colchicine or thiocolchicine dimer comprises two thiocolchicine subunits (hereinafter referred to as “thiocolchicine dimer”). In some embodiments, the colchicine or thiocolchicine dimer comprises two colchicine subunits (herein after referred to as “colchicine dimer”).

In some embodiments, the colchicine or thiocolchicine dimer is a compound of formula (I):

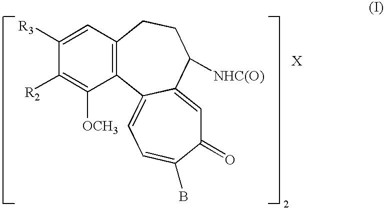

wherein the B in each subunit is either a methoxy or a methylthio group, R₂ is methoxy, hydroxyl, or methylenedioxy when taken together with R₃, R₃ is methoxy, hydroxyl, or methylenedioxy when taken together with R₂, and X is a linking group.

A wide variety of cross-linking groups can be used to introduce the linking group X. One of skill in the art will recognize that the colchine or thiocolchicine monomer components of the dimer have a single reactive amino group; should any other reactive (nucleophilic) groups be present on the intermediates, they can be readily protected using groups well-known in the art. For examples of protecting groups, see, for example, Greene, T. W., and P. G. M. Wuts, Protective Groups in Organic Synthesis, 3^{rd} edition, Wiley: New York, 1999, the contents of which are hereby incorporated herein by reference in their entirety. Thus, a wide variety of cross-linking groups reactive with the amine functionality can be employed.

In some embodiments, the linking group X comprises at least one carbon atom. For example, the commercially available (Sigma-Aldrich) reagent malonyl chloride, Cl—C(O)—CH₂—C(O)—Cl, can be used to form a colchicine dimer where the X group is —CH₂—. Similarly, other diacyl chlorides of varying lengths can be used to form X groups of desired length. For example, in formula (II), when n=8 and Y is CH₂, the commercially available (Sigma-Aldrich) reagent dodecanedioyl dichloride, Cl—C(O)—(CH₂)₁₀—C(O)—Cl, can be used to synthesize the dimer where the X group is —(CH₂)₁₀—. For groups where Y is NH and n=1, the reagent 3-isocyanatopropanoyl chloride (Organic Syntheses, Coll. Vol. 6, p. 715 (1988); Vol. 59, p. 195 (1979)) can be used to synthesize the linking group X when X is —NH—CH₂CH₂—. Other well-known cross-linking reagents can be used to generate the X linker. One of skill in the art is directed to Wong, Shan S., Chemistry of Protein Conjugation and Cross Linking, CRC Press: Boca Raton, 1991, in particular, Chapter 2, Section IV(B)., pp. 33-38, directed towards amino-group reactive agents; Chapter 4, Section II, pp. 75-103, directed towards amino-group reactive cross-linkers; and Chapter 7, pp. 209-220, directed toward procedures and analysis for cross-linking reactions for reagents and procedures suitable for cross-linking amino-containing compounds. The entire contents of the aforementioned Wong reference, and particularly the specific sections enumerated, are hereby incorporated herein by reference.

In some embodiments, the colchicine or thiocolchicine dimer is a compound of the formula (II):

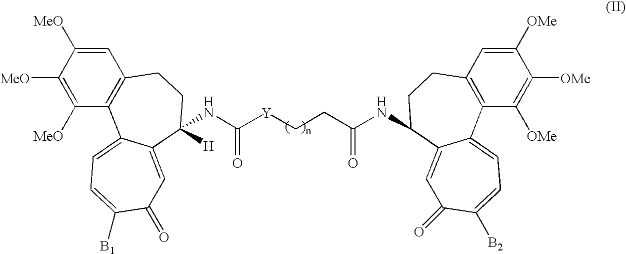

wherein B₁ is a methoxy or a methylthio group, B₂ is a methoxy or a methylthio group, n is an integer from 0 to 8, Y is a CH₂ group or, when n is 1, can also be a group of formula NH.

In some embodiments, n is any of (and in some embodiments selected from the group consisting of) 0, 1, 2, 3, 4, 5, 6, 7, or 8. In some embodiments, n is 1. In some embodiments, n is 1 and Y is NH. In some embodiments, n is 2.

In some embodiments, both B₁ and B₂ are methoxy groups. In some embodiments, both B₁ and B₂ are methylthio groups. In some embodiments, B₁ is methoxy group and B₂ is methylthio group. In some embodiments, B₁ is methylthio group and B₂ is methoxy group. In some embodiments, the colchicine or thiocolchicine dimer is any of (and in some embodiments selected from the group consisting of): IDN5404, IDN5676, IDN5800, and IDN5801.

In some embodiments, the compound is thiocolchicine dimer IDN5404. IDN5404 is a compound of formula (III):

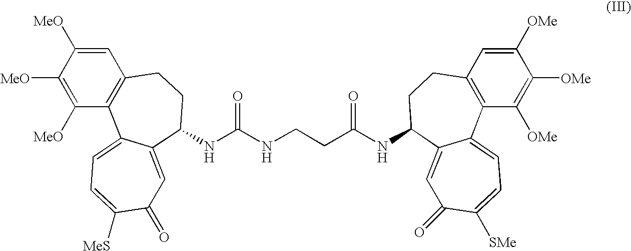

In some embodiments, the compound is thiocolchicine dimer IDN5676. IDN5676 is a compound of formula (IV):

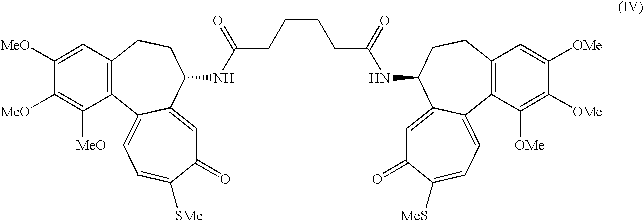

### Biocompatible Polymers and Carrier Protein

In some embodiments, the colchicine or thiocolchicine dimer compositions described herein further comprise a biocompatible polymer such as a carrier protein.

As used herein, the term “biocompatible” describes a substance that does not appreciably alter or affect in any adverse way, the biological system into which it is introduced. Biocompatible polymer includes naturally-occurring or synthetic biocompatible materials such as proteins, peptides, polynucleotides, polysaccharides (e.g., starch, cellulose, dextrans, alginates, chitosan, pectin, hyaluronic acid, and the like), and lipids. Suitable biocompatible polymers include, for example, naturally occurring or synthetic proteins such as albumin, insulin, hemoglobin, lysozyme, immunoglobulins, α-2-macroglobulin, fibronectin, vitronectin, fibrinogen, casein and the like, as well as combinations of any two or more thereof. Synthetic polymers include, for example, polyalkylene glycols (e.g., linear or branched chain), polyvinyl alcohol, polyacrylates, polyhydroxyethyl methacrylate, polyacrylic acid, polyethyloxazoline, polyacrylamides, polyisopropyl acrylamides, polyvinylpyrrolidone, polylactide/glycolide and the like, and combinations thereof.

The term “proteins” refers to polypeptides or polymers of amino acids of any length (including full length or fragments), which may be linear or branched, comprise modified amino acids, and/or be interrupted by non-amino acids. The term also encompasses an amino acid polymer that has been modified naturally or by intervention; for example, disulfide bond formation, glycosylation, lipidation, acetylation, phosphorylation, or any other manipulation or modification. Also included within this term are, for example, polypeptides containing one or more analogs of an amino acid (including, for example, unnatural amino acids, etc.), as well as other modifications known in the art. The proteins described herein may be naturally occurring, i.e., obtained or derived from a natural source (such as blood), or synthesized (such as chemically synthesized or by synthesized by recombinant DNA techniques).

Examples of suitable carrier proteins include proteins normally found in blood or plasma, which include, but are not limited to, albumin, immunoglobulin including IgA, lipoproteins, apolipoprotein B, alpha-acid glycoprotein, beta-2-macroglobulin, thyroglobulin, transferrin, fibronectin, factor VII, factor VIII, factor IX, factor X, and the like. In some embodiments, the carrier protein is a non-blood protein, such as casein, α-lactalbumin, and β-lactoglobulin. The carrier proteins may either be natural in origin or synthetically prepared. In some embodiments, the carrier protein is an albumin, such as human serum albumin. Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977)) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary animals (including domestic pets and agricultural animals).

Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (1981), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (1992), and Carter et al., Adv. Protein. Chem., 45, 153-203 (1994)).

To provide an example, carrier proteins are further described below. It is understood that this description generally applies to biocompatible polymers.

The carrier protein (such as albumin) in the composition generally serves as a carrier for the colchicine or thiocolchicine dimer, i.e., the carrier protein in the composition makes the colchicine or thiocolchicine dimer more readily suspendable in an aqueous medium or helps maintain the suspension as compared to compositions not comprising a carrier protein. This can avoid the use of toxic solvents for solubilizing the colchicine or thiocolchicine dimer, and thereby can reduce one or more side effects caused by those toxic solvent. In some embodiments, the composition is substantially free of surfactants, i.e., the amount of surfactant in the composition is not sufficient to cause one or more side effect(s) in an individual when the composition is administered to the individual. In some embodiments, the composition is free of surfactants.

In some embodiments, the carrier protein is associated with the colchicine or thiocolchicine dimer, i.e., the composition comprises carrier protein-associated colchicine or thiocolchicine dimer. “Association” or “associated” is used herein in a general sense and refers to the carrier protein affecting a behavior and/or property of the colchicine or thiocolchicine dimer in an aqueous composition. For example, the carrier protein and the colchicine or thiocolchicine dimer are considered as being “associated” if the carrier protein makes the colchicine or thiocolchicine dimer more readily suspendable in an aqueous medium as compared to a composition without the carrier protein. As another example, the carrier protein and the colchicine or thiocolchicine dimer is associated if the carrier protein stabilizes the colchicine or thiocolchicine dimer in an aqueous suspension. For example, the carrier protein and the colchicine or thiocolchicine dimer can be present in a particle or a nanoparticle, which are further described herein.

A colchicine or thiocolchicine dimer is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

The carrier protein and the colchicine or thiocolchicine dimer in the composition can be associated in various manners. For example, in some embodiments, the carrier protein is in admixture with the colchicine or thiocolchicine dimer. In some embodiments, the carrier protein encapsulates or entraps the colchicine or thiocolchicine dimer. In some embodiments, the carrier protein is bound (such as non-covalently bound) to the colchicine or thiocolchicine dimer. In some embodiments, the composition may exhibit one or more of the above aspects.

In some embodiments, the composition comprises particles (such as microparticles or nanoparticles) comprising (in various embodiments consisting essentially of) a colchicine or thiocolchicine dimer and a carrier protein (such as albumin). In some embodiments, the colchicine or thiocolchicine dimer is coated with the carrier protein (such as albumin). In some embodiments, the coating consists essentially of or consists of the carrier protein (such as albumin). In some embodiments, at least a portion of the carrier protein in the particle portion of the composition is crosslinked (for example crosslinked by disulfide bonds). In some embodiments, the colchicine or thiocolchicine dimers in the particles are amorphous. In some embodiments, the particles are substantially free of polymeric core materials.

In some embodiments, the composition comprises more than about 50% (for example more than about any of 60%, 70%, 80%, 90%, or 95%) of the colchicine or thiocolchicine dimer in particle form. In some embodiments, the weight percentage of the colchcine or thiocochicine dimer in the particle portion of the composition is at least about any of 50%, 60%, 70%, 80%, 90%, or 95% of the total weight of the particle portion of the composition.

In some embodiments, the composition comprises nanoparticles (i.e., particles with an average or mean diameter of no greater than about 1000 nanometers (nm)), such as no greater than about any of 900, 800, 700, 600, 500, 400, 300, 200, 100, 80, 60, or 50 nm. In some embodiments, the nanoparticles are spherical. In some embodiments, the nanoparticles are non-spherical. In some embodiments, the average or mean diameters of the nanoparticles in the composition is no greater than about 200 nm (including for example no greater than about 100 nm). In some embodiments, the average or mean diameter of the nanoparticles in the composition is between about 20 to about 400 nm. In some embodiments, the average or mean diameter of the nanoparticles is between about 40 to about 200 nm. In some embodiments, the nanoparticles are sterile-filterable.

The particles (such as microparticles or nanoparticles) described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

The amount of carrier protein in the composition described herein will vary depending on the pharmaceutical agent and other components in the composition. In some embodiments, the composition comprises a carrier protein in an amount that is sufficient to stabilize the colchicine or thiocolchicine dimer in an aqueous suspension, for example, in the form of a stable colloidal suspension (such as a stable suspension of microparticles or nanoparticles). In some embodiments, the carrier protein is in an amount that reduces the sedimentation rate of the colchicine or thiocolchicine dimer in an aqueous medium. For particle-containing compositions, the amount of the carrier protein also depends on the size and density of particles of the colchicine or thiocolchicine dimer.

In some embodiments, the carrier protein is present in an amount that is sufficient to stabilize the colchicine or thiocolchicine dimer in an aqueous suspension at a certain concentration. For example, the concentration of the colchicine or thiocolchicine dimer in the composition is about 0.01 to about 100 mg/ml, including for example any of about 0.01 to about 50 mg/ml, about 0.1 to about 50 mg/ml, about 1 to about 10 mg/ml, about 2 mg/ml to about 8 mg/ml, about 4 to about 6 mg/ml, about 5 mg/ml. In some embodiments, the concentration of the colchicine or thiocolchicine dimer is at least about any of 0.01 mg/ml, 0.03 mg/ml, 0.05 mg/ml, 0.08 mg/ml, 0.1 mg/ml, 0.3 mg/ml, 0.5 mg/ml, 0.8 mg/ml, 1 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the carrier protein is present in an amount that avoids use of surfactants, so that the composition is free or substantially free of surfactant.

In some embodiments, the composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of carrier protein. In some embodiments, the composition, in liquid form, comprises about 0.5% to about 5% (w/v) of carrier protein.

In some embodiments, the weight ratio of carrier protein, e.g., albumin, to the colchicine or thiocolchicine dimer is such that a sufficient amount of colchicine or thiocolchicine dimer binds to, or is transported by, the cell. In some embodiments, the weight ratio of carrier protein, e.g., albumin, to colchicine or thiocolchicine dimer (w/w) is any of about 0.01:1 to about 100:1, about 0.02:1 to about 50:1, about 2:1 to about 30:1, about 0.05:1 to about 20:1, about 0.1:1 to about 20:1, about 1:1 to about 18:1, about 2:1 to about 15:1, about 3:1 to about 12:1, about 4:1 to about 10:1, about 8:1 to about 10:1, about 5:1 to about 9:1, or about 9:1. In some embodiments, the carrier protein (such as albumin) to colchicine or thiocolchicine dimer weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, 3:1 or less, or 2:1 or less.

In some embodiments, the composition comprises particles (such as microparticles or nanoparticles) comprising (in various embodiments consisting essentially of) thiocolchicine dimer (such as IDN5404 or IDN5676) and albumin. The particles (such as microparticles or nanoparticles) may have an average or mean diameter of no greater than about 10, 9, 8, 7, 6, 5, 4, 3, 2, or 1 micron. In some embodiments, the particle is a nanoparticle, i.e., a particle that is less than about 1000 nanometers (nm). For example, the nanoparticle may be no greater than about any of 900, 800, 700, 600, 500, 400, 300, 200, 100, 80, or 50 nm. In some embodiments, the average or mean diameters of the nanoparticles is no greater than about 200 nm (including for example no greater than about 100 nm). In some embodiments, the average or mean diameter of the nanoparticles is between about 20 to about 400 nm. In some embodiments, the average or mean diameter of the nanoparticles is between about 40 to about 200 nm. In some embodiments, the particles are sterile-filterable.

In some embodiments, the thiocolchicine dimer (such as IDN5404 or IDN5676) is coated with albumin. In some embodiments, the weight ratio of albumin to thiocolchicine dimer (such as IDN5404 or IDN5676) (w/w) is any of about 0.01:1 to about 100:1, about 0.02:1 to about 50:1, about 2:1 to about 30:1, about 0.05:1 to about 20:1, about 0.1:1 to about 20:1, about 1:1 to about 18:1, about 2:1 to about 15:1, about 3:1 to about 12:1, about 4:1 to about 10:1, about 8:1 to about 10:1, about 5:1 to about 9:1, and about 9:1. In some embodiments, the albumin to thiocolchicine dimer weight ratio is less than about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, 3:1 or less, and 2:1 or less.

In some embodiments, the particles (such as microparticles or nanoparticles) comprising thiocolchicine dimer (such as IDN5404 or IDN5676) and albumin are suspended in an aqueous medium (such as an aqueous medium containing the albumin). For example, the composition can be a colloidal suspension of thiocolchicine dimer (such as IDN5404 or IDN5676)-containing particles (such as microparticles or nanoparticles). In some embodiments, the composition is a dry composition (such as lyophilized composition) that can be reconstituted to an aqueous suspension of thiocolchicine dimer (such as IDN5404 or IDN5676)-containing particles. In some embodiments, the concentration of thiocolchicine dimer (such as IDN5404 or IDN5676) in the composition is between about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, and about 5 mg/ml. In some embodiments, the concentration of IDN5404 or IDN5676 is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml.

In some embodiments, the composition comprises a nanoparticle formulation of IDN5404 or IDN5676 (hereinafter referred to as Nab-5404 or Nab-5676). Nab-IDN5404 and Nab-5676 are nanoparticle formulation of IDN5404 and IDN5676, respectively, stabilized by human serum albumin. These nanoparticle formulations can be generated by methods described in U.S. Pat. No. 5,916,596 and U.S. Pat. App. Pub. No. 2005/0004002. When dispersed in a suitable aqueous medium such as 0.9% sodium chloride injection or 5% dextrose injection, Nab-5404 (or Nab-5676) forms a stable colloidal suspension of thiocolchicine dimer. The size (i.e., average or mean diameter) of the particles in the colloidal suspension may range from 20 nm to 8 microns with a preferred range of about 20-400 nm. Since HSA is freely soluble in water, Nab-5404 (or Nab-5676) can be reconstituted in a wide range of concentrations ranging from dilute (0.1 mg/ml IDN5404 or IDN5676) to concentrated (20 mg/ml IDN5404 or IDN5676), including for example about 2 mg/ml to about 8 mg/ml, about 5 mg/ml. In some embodiments, the IDN5404 or IDN5676 concentration is about any of 1, 2, 3, 4, 5, 6, 7, 8, 9, 10, 11, 12, 13, 14, 15, 16, 17, 18, 19, and 20 mg/ml.

### Pharmaceutical Compositions, Unit Doses, and Kits

Also provided herein are pharmaceutical compositions comprising a colchicine or thiocolchicine dimer. The pharmaceutical composition may be suitable for a variety of modes of administration described herein, including for example systemic or local administration. The pharmaceutical composition can be in the form of eye drops, injectable solutions, or in a form suitable for inhalation (either through mouth or nose), or oral administration. The pharmaceutical compositions described herein can be packaged in single unit dosages or in multidosage forms. In some embodiments, the composition is suitable for administration to a human. In some embodiments, the composition is suitable for administration to a mammal such as, in the veterinary context, domestic pets and agricultural animals. There are a wide variety of suitable formulations of the composition (see, e.g., U.S. Pat. Nos. 5,916,596 and 6,096,331).

The pharmaceutical compositions may comprise a colchicine or thiocolchicine dimer and a pharmaceutically acceptable carrier suitable for delivery to the eye and/or intraarterial injection. The pharmaceutical compositions may further comprise carrier proteins, such as carrier proteins described herein. For example, in some embodiments, the pharmaceutical composition comprises a colchicine or thiocolchicine dimer, a carrier protein, and a pharmaceutically acceptable carrier suitable for delivery to the eye. For example, the pharmaceutically acceptable carrier can be suitable for any one or more of: intraocular injection, periocular injection, subretinal injection, intravetreal injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, the pharmaceutically acceptable carrier is suitable for intraocular injection. In some embodiments, the pharmaceutically acceptable carrier is suitable for topical application to the eye.

In some embodiments, the pharmaceutical composition comprises a colchicine or thiocolchicine dimer, a carrier protein, and a pharmaceutically acceptable carrier suitable for intraarterial injection.

The pharmaceutical compositions are generally formulated as sterile and substantially isotonic compositions. The pharmaceutical compositions described herein are generally in full compliance with all Good Manufacturing Practice (GMP) regulations of the U.S. Food and Drug Administration. In some embodiments, the composition is free of pathogen. For injection, the pharmaceutical composition can be in the form of liquid solutions, for example, in physiologically compatible buffers such as Hank's solution or Ringer's solution. The pharmaceutical composition can also be in a solid form and redissolved or resuspended immediately prior to use. Lyophilized compositions are also included.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

The present invention in some embodiments provides composition comprising a colchicine or thiocolchicine dimer, optionally a carrier protein, and a pharmaceutically acceptable carrier suitable for administration to the eye. Such pharmaceutical carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Suitable pharmaceutical excipients include starch, glucose, lactose, sucrose, gelatin, malt, rice, sodium stearate, glycerol monostearate, glycerol, propylene, water, and the like. The pharmaceutical composition, if desired, can also contain minor amounts of wetting or emulsifying agents, or pH buffering agents. The components of the composition may be encased in polymers or fibrin glues to provide controlled release of the molecule. These compositions can take the form of solutions, suspensions, emulsions, ointment, gel, or other solid or semisolid compositions, and the like. The compositions typically have a pH in the range of 4.5 to 8.0. The compositions must also be formulated to have osmotic values that are compatible with the aqueous humor of the eye and ophthalmic tissues. Such osmotic values will generally be in the range of from about 200 to about 400 milliosmoles per kilogram of water (“mOsm/kg”), but will preferably be about 300 mOsm/kg.

In some embodiment, the composition is formulated in accordance with routine procedures as a pharmaceutical composition adapted for injection intravenously, introperitoneally, or intravitreously. Typically, compositions for injection are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions may further comprise additional ingredients, for example preservatives, buffers, tonicity agents, antioxidants and stabilizers, nonionic wetting or clarifying agents, viscosity-increasing agents, and the like.

Suitable preservatives for use in a solution include polyquarternium-1, benzalkonium chloride, thimerosal, chlorobutanol, methyl paraben, propyl paraben, phenylethyl alcohol, edetate disodium, sorbic acid, benzethonium chloride, and the like. Typically (but not necessarily) such preservatives are employed at a level of from 0.001% to 1.0% by weight.

Suitable buffers include boric acid, sodium and potassium bicarbonate, sodium and potassium borates, sodium and potassium carbonate, sodium acetate, sodium biphosphate and the like, in amounts sufficient to maintain the pH at between about pH 4.5 to about 9, including for example about pH 5 to about pH 8, about pH 6 to about pH 8, or about pH 7 to about pH 7.5.

Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfate, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

The use of viscosity enhancing agents to provide topical compositions with viscosities greater than the viscosity of simple aqueous solutions may be desirable to increase ocular absorption of the active compounds by the target tissues or increase the retention time in the eye. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

The compositions described herein can also include other agents, excipients, or stabilizers to improve properties of the composition. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

Also provided are unit dosage forms of colchicine or thiocolchicine dimer compositions. These unit dosage forms can be stored in a suitable packaging in single or multiple unit dosages and may also be further sterilized and sealed. The term “unit dosage form” refers to a physically discrete unit suitable as unitary dosages for an individual, each unit containing a predetermined quantity of active material calculated to produce the desired therapeutic effect, in association with a suitable pharmaceutical carrier, diluent, or excipient. These unit dosage forms can be stored in a suitable packaging in single or multiple unit dosages and may also be further sterilized and sealed. In some embodiments, there is provided a unit dosage form of colchicine or thiocolchicine dimer (and optionally a carrier protein) that is suitable for intraocular injection. In some embodiments, there is provided a unit dosage form of colchicine or thiocolchicine dimer (and optionally a carrier protein) that is suitable for topical application to the eye. In some embodiments, there is provided a unit dosage form of colchicine or thiocolchicine dimer (and optionally a carrier protein) that is suitable for intraarterial injection.

In some embodiments, there is provided a unit dosage form comprising colchicine or thiocolchicine dimer in an amount that is effective in inhibiting angiogenesis, wherein the amount of the composition is insufficient to induce significant cytotoxicity in the individual. In some embodiments, there is provided a unit dosage form comprising colchicine or thiocolchicine dimer in an amount that is effective in treating cancer, wherein the amount of colchicine or thiocolchicine dimer in the composition per administration is less than about any of 1%, 2%, 3%, 4%, 5%, 6%, 7%, 8%, 9%, 10%, 11%, 12%, 13%, 14%, 15%, 20%, 25%, 30%, 40%, or 50% of the corresponding MTD for the colchicine or thiocolchicine dimer composition.

The unit dosage form may comprise a colchicine or thiocolchicine dimer in an amount that is sufficient to deliver about 0.25 mg/m² to about 100 mg/m² colchicine or thiocolchicine dimer to a subject, including for example any of about 0.5 mg/m² to about 50 mg/m², about 1 mg/m² to about 15 mg/m², about 2 mg/m² to about 10 mg/m², about 3 mg/m² to about 8 mg/m². In some embodiments, the unit dosage form comprises colchicine or thiocolchicine dimer in an amount that is sufficient to delivery any of about 0.25 mg/m² to about 0.5 mg/m², about 0.5 mg/m² to about 1 mg/m², about 1 mg/m² to about 2 mg/m², about 2 mg/m² to about 3 mg/m², about 3 mg/m² to about 4 mg/m², about 4 mg/m² to about 5 mg/m², about 5 mg/m² to about 6 mg/m², about 6 mg/m² to about 7 mg/m², about 7 mg/m² to about 8 mg/m², about 8 mg/m² to about 9 mg/m², about 9 mg/m² to about 10 mg/m², about 10 mg/m² to about 50 mg/m², about 50 mg/m² to about 100 mg/m² colchicine or thiocolchicine dimer to a subject.

In some embodiments, the unit dosage form comprises about 0.05 mg to about 200 mg colchicine or thiocolchicine dimer. For example, a unit dosage containing from about 0.1 mg to about 50 mg, about 0.2 mg to about 50 mg, about 0.5 mg to about 30 mg, about 1 mg to about 20 mg, or about 15 mg of the colchicine or thiocolchicine dimer is contemplated. Other exemplary amounts of colchicine or thiocolchicine dimer in a unit dosage form include, but are not limited to, about 0.05 mg to about 0.1 mg, about 0.1 mg to about 0.2 mg, about 0.2 mg to about 0.3 mg, about 0.3 mg to about 0.4 mg, about 0.4 mg to about 0.5 mg, about 0.5 mg to about 1 mg, about 1 mg to about 5 mg, about 5 to about 10 mg, about 10 mg to about 15 mg, about 15 mg to about 20 mg, about 20 mg to about 25 mg, about 25 mg to about 50 mg, or about 50 mg to about 100 mg. In some embodiments, the concentration of colchicine or thiocolchicine dimer in the composition is less than about any of 0.1 mg/ml, 0.2 mg/ml, 0.5 mg/ml, 1 mg/ml, 2 mg/ml, 3 mg/ml, 4 mg/m, or 5 mg/ml.

Also provided are articles of manufacture comprising the compositions, formulations, and unit dosages described herein in suitable packaging for use in the methods described herein. Suitable packaging for compositions (such as ophthalmic compositions) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present invention also provides kits comprising compositions (or unit dosages forms and/or articles of manufacture) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein. For example, in some embodiments, the kit comprises a colchicine or thiocolchicine dimer, a carrier protein, a pharmaceutically acceptable carrier suitable for intraocular injection, and one or more of: a buffer, a diluent, a filter, a needle, a syringe, and a package insert with instructions for performing intraocular injection. In some embodiments, the pharmaceutical composition comprises a colchicine or thiocolchicine dimer, a carrier protein, a pharmaceutically acceptable carrier suitable for intraarterial injection, and one or more of: a buffer, a diluent, a filter, a needle, a syringe, and a package insert with instructions for performing intraarterial injection.

### Methods of Making Nanoparticles Compositions Comprising Colchicine or Thiocolchicine Dimers and Carrier Proteins

Nanoparticle compositions comprising colchicine or thiocolchicine dimmers and carrier proteins can be prepared by methods known in the art. For example, nanoparticles containing poorly water soluble pharmaceutical agents and carrier proteins (e.g., albumin) can be prepared under conditions of high shear forces (e.g., sonication, high pressure homogenization, or the like). These methods are disclosed in, for example, U.S. Pat. Nos. 5,916,596; 6,506,405; and 6,537,579 and also in U.S. Pat. Pub. No. 2005/0004002A1, which are each hereby incorporated by reference in their entireties.

Briefly, the colchicine or thiocolchicine dimer is dissolved in an organic solvent. Suitable organic solvents include, for example, ketones, esters, ethers, chlorinated solvents, and other solvents known in the art. For example, the organic solvent can be methylene chloride, chloroform/ethanol, or chloroform/t-butanol (for example with a ratio (w/w) of about any of 1:9, 1:8, 1:7, 1:6, 1:5, 1:4, 1:3, 1:2, 1:1, 2:1, 3:1, 4:1, 5:1, 6:1, 7:1, 8:1, or 9:1 or with a ratio (w/w) of about any of 3:7, 5:7, 4:6, 5:5, 6:5, 8:5, 9:5, 9.5:5, 5:3, 7:3, 6:4, or 9.5:0.5). The solution is added to a carrier protein (e.g., human serum albumin). The mixture is subjected to high pressure homogenization (e.g., using an Avestin, APV Gaulin, Microfluidizer™ such as a Microfluidizer™ Processor M-110EH from Microfluidics, Stansted, or Ultra Turrax homogenizer). The emulsion may be cycled through the high pressure homogenizer for between about 2 to about 100 cycles, such as about 5 to about 50 cycles or about 8 to about 20 cycles (e.g., about any of 8, 10, 12, 14, 16, 18 or 20 cycles). The organic solvent can then be removed by evaporation utilizing suitable equipment known for this purpose, including, but not limited to, rotary evaporators, falling film evaporators, wiped film evaporators, spray driers, and the like that can be operated in batch mode or in continuous operation. The solvent may be removed at reduced pressure (such as at about any of 25 mm Hg, 30 mm Hg, 40 mm Hg, 50 mm Hg, 100 mm Hg, 200 mm Hg, or 300 mm Hg). The amount of time used to remove the solvent under reduced pressure may be adjusted based on the volume of the formulation. For example, for a formulation produced on a 300 mL scale, the solvent can be removed at about 1 to about 300 mm Hg (e.g., about any of 5-100 mm Hg, 10-50 mm Hg, 20-40 mm Hg, or 25 mm Hg) for about 5 to about 60 minutes (e.g., about any of 7, 8, 9, 10, 11, 12, 13, 14, 15 16, 18, 20, 25, or 30 minutes).

If desired, human albumin solution may be added to the dispersion to adjust the human serum albumin to colchicine or thiocolchicine dimer ratio or to adjust the concentration of the colchicine or thiocolchicine dimer in the dispersion. For example, human serum albumin solution (e.g., 25% w/v) can be added to adjust the human serum albumin to a colchicine or thiocolchicine dimer ratio (w/w) to about any of 18:1, 15:1, 14:1, 13:1, 12:1, 11:1, 10:1, 9:1, 8:1, 7.5:1, 7:1, 6:1, 5:1, 4:1, or 3:1. For example, human serum albumin solution (e.g., 25% w/v) or another solution is added to adjust the concentration of a colchicine or thiocolchicine dimer in the dispersion to about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml. The dispersion may be serially filtered through multiple filters, such as a combination of 1.2 μm and 0.8/0.2 μm filters; the combination of 1.2 μm, 0.8 μm, 0.45 μm, and 0.22 μm filters; or the combination of any other filters known in the art. The dispersion obtained can be further lyophilized. The nanoparticle compositions may be made using a batch process or a continuous process (e.g., the production of a composition on a large scale).

If desired, an antimicrobial agent, sugar, and/or stabilizing agent can also be included in the composition. This additional agent can either be admixed with the colchicine or thiocolchicine dimer and/or the carrier protein during preparation of the colchicine or thiocolchicine dimer/carrier protein composition, or added after the colchicine or thiocolchicine dimer/carrier protein composition is prepared. In some embodiments, the agent is admixed with the colchicine or thiocolchicine dimer/carrier protein composition prior to lyophilization. In some embodiments, the agent is added to the lyophilized pharmaceutical agent/carrier protein composition. In some embodiments when the addition of the agent changes the pH of the composition, the pH in the composition are generally (but not necessarily) adjusted to a desired pH. Exemplary pH values of the compositions include, for example, in the range of about 5 to about 8.5. In some embodiments, the pH of the composition is adjusted to no less than about 6, including for example no less than any of about 6.5, 7, or 8 (e.g., about 8).

Unless defined otherwise, the meanings of all technical and scientific terms used herein are those commonly understood by one of skill in the art to which this invention belongs. Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

### EXAMPLES

### Example 1

### In vitro Cytotoxity Activity of Nab-5404 and Nab-5676

The cytotoxic activities of Nab-5404 and Nab-5676 were evaluated in vitro using MX-1 breast carcinoma, HepG2 hepatoma, HT-29 colon carcinoma cell lines and normal primary rat hepatocytes. Cells were exposed to Nab-5404 and Nab-5676 for 72 hours at 37° C. across a range of increasing concentrations. Viability was analyzed using a Cell Titer Blue cell viability assay (Promega, Madison, Wis.). IC50s were calculated with a one-phase exponential decay equation using Prism software (GraphPad, San Diego, Calif.).

Nab-5404 demonstrated cytotoxic activity against HepG2 hepatoma cells and primary rat hepatocytes, with IC50s of 16 and 9 μg/ml respectively. Both Nab-5404 and Nab-5676 were moderately cytotoxic against MX-1 cells, with IC50s of 43 and 54 μg/ml respectively. Activity of Nab-5404 and Nab-5676 against HT-29 cells was low with IC50s of 110 and 149 μg/ml, respectively. Nab-5676 showed little or no cytotoxic activity in HepG2 cells or primary rat hepatocytes. Results are summarized in Table 1.

TABLE 1

| | Nab-5404 | Nab-5676 |
|---|---|---|
| Cell Line | IC50 (μg/ml) | IC50 (μg/ml) |
| MX-1 breast carcinoma | 43 | 54 |
| HepG2 hepatoma | 16 | 125 |
| HT-29 colon carcinoma | 110 | 149 |
| Primary rat hepatocytes | 9 | Inactive |

### Example 2

### Anti-microtubule Activity of Nab-5404 and Nab-5676

The microtubule depolymerization activities of Nab-5404 and Nab-5676 were tested using the MX-1 breast carcinoma cell line. MX-1 cells were seeded on cover slips and treated with Nab-5404 or Nab-5676 for 2 hours at 37° C. across a concentration range of 0.01 to 100 μg/ml. After incubation, the cells were fixed and stained for tubulin and actin. Tubulin was stained with monoclonal anti-tubulin antibody and actin was stained with fluorescein-labeled phalloidin. The microtubule network was visualized and analyzed using ImagePro Software (MediaCybernetics, Inc., Silver Spring Md.). IC50s for Nab-5404 and Nab-5676 activity on microtubule destabilization were calculated using Prism software (GraphPad, San Diego, Calif.).

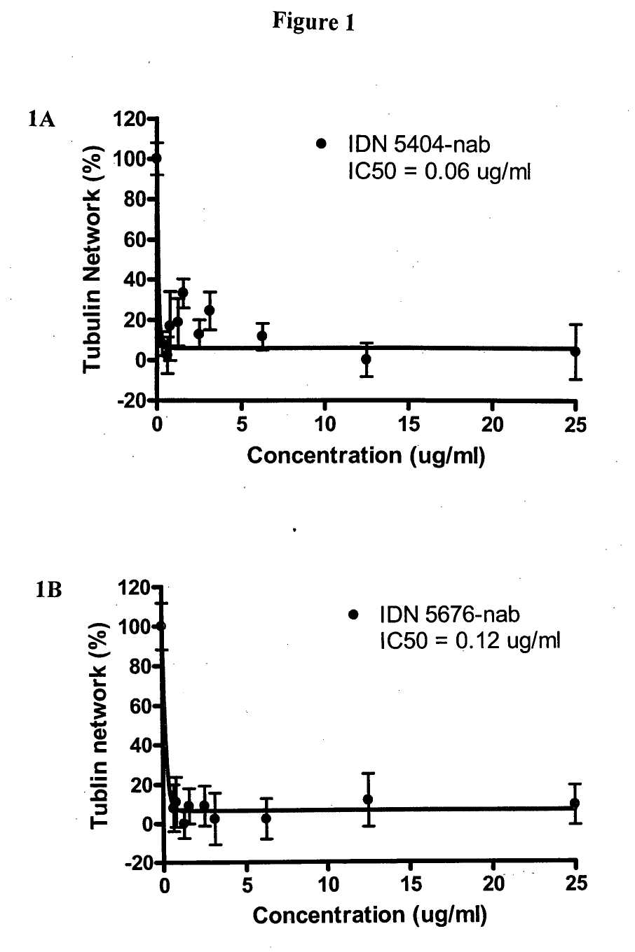

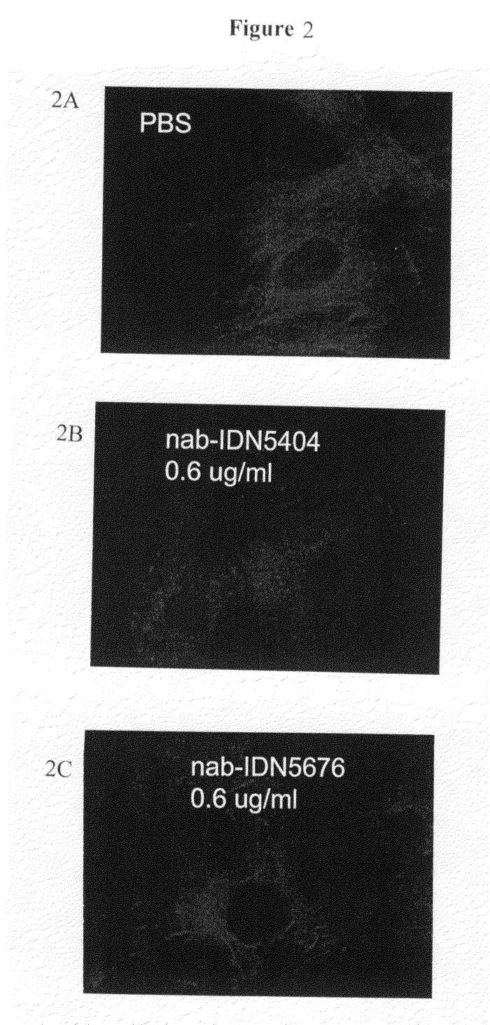

Both Nab-5404 and Nab-5676 exhibited potent microtubule depolymerization activity with calculated IC50s of 0.06 μg/ml and 0.12 μg/ml, respectively (FIGS. 1A and 1B). Even at the lowest concentration tested, 0.6 μg/ml, the microtubule network was completely destroyed after a two-hour incubation with Nab-5404 or Nab-5676 (FIGS. 2A-2C). In contrast, actin bundles were not affected by Nab-5404 or Nab-5676 at any drug concentration.

### Example 3

### Anti-Angiogenic Activity of Nab-5404 and Nab-5676

To study the effect of Nab-5404 and Nab-5676 on angiogenesis, the compounds were evaluated in a microvessel formation assay using a TCS CellWorks AngioKit model (TCS CellWorks Ltd., Botolph Claydon, Buckingham UK). The AngioKit model uses human endothelial cells co-cultured with other human cells. The endothelial cells initially form small islands within the culture matrix, then begin to proliferate and then enter a migratory phase during which they move through the matrix to form threadlike tubule structures. These gradually join up to form a network of tubules which closely resemble a capillary bed (at day 9-11). The tubules stain positive for von Willebrand's factor, CD31 (PECAM-1) and ICAM-2.

In addition to Nab-5404 and Nab-5676, a known vascular targeting agent, combretastatin 4-phosphate (CA4P), was tested for in vitro anti-angiogenic activity. The TCS CellWorks AngioKit model was used according to the manufacturer's instructions. As outlined above, human endothelial cells were co-cultured with human fibroblast cells in a 24 well plate and exposed to Nab-5404, Nab-5676 or CA4P across a range of concentrations (0.01 to 100 μg/ml). After a 11 or 12 day incubation, tubules were visualized by fixing the cells and staining using a monoclonal antibody against CD31, a secondary antibody conjugate and a colored substrate. The length of the tubules was analyzed using ImagePro software and the IC50 of each compound was calculated using Prism software.

Both Nab-5404 and Nab-5676 inhibited tubule formation and disrupted established tubules demonstrating anti-angiogenic activity. In the first experiment, the cells were treated with the compositions on day 1 and stained on day 12 for analysis of inhibition of tubule formation. In the second experiment, the cells were treated on day 8 and stained on day 11, while in the third experiment, the cells were treated on day II and stained on day 12 which allowed for analysis of the disruption of established tubules.

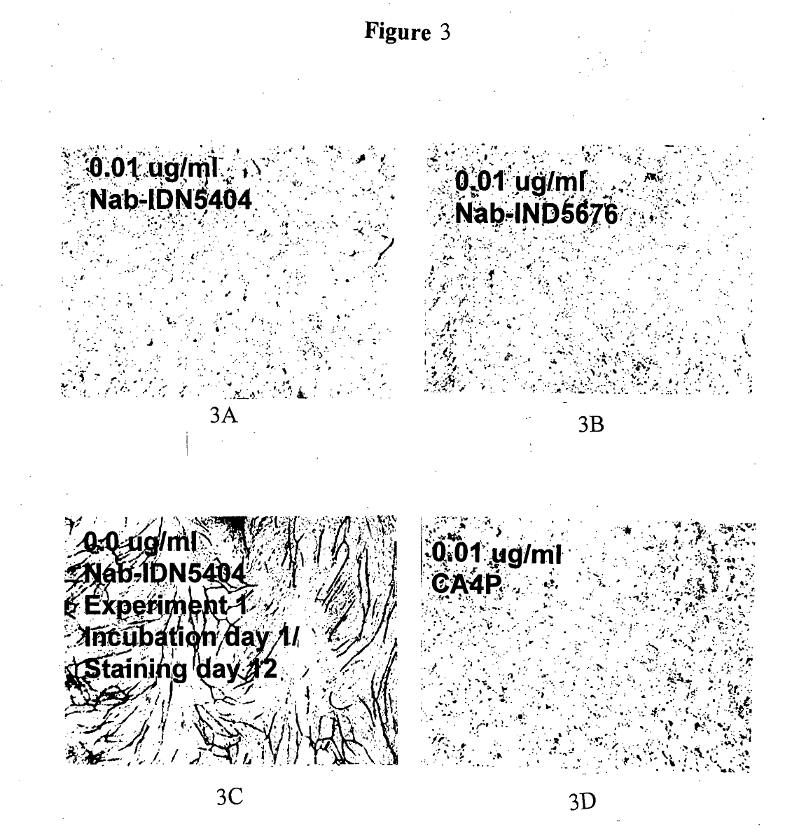

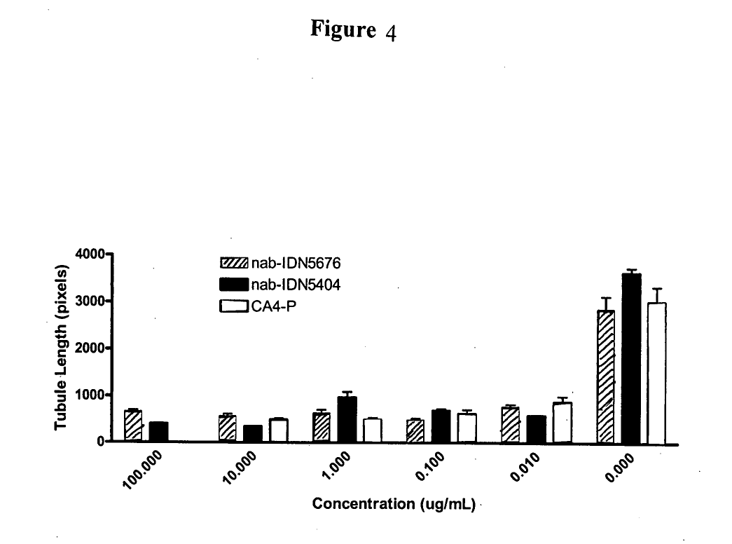

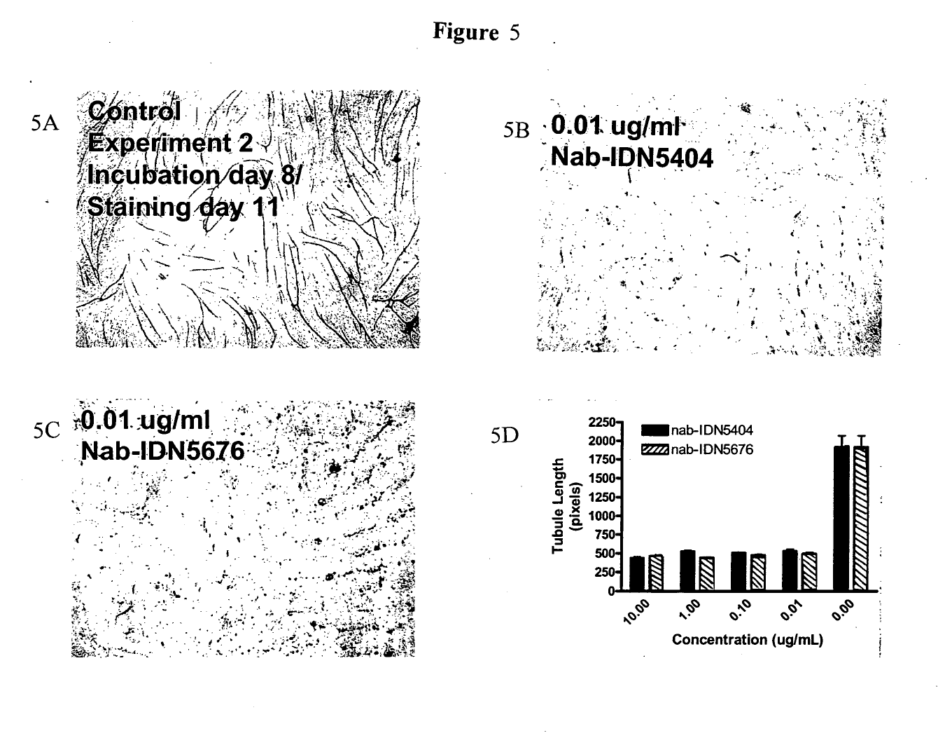

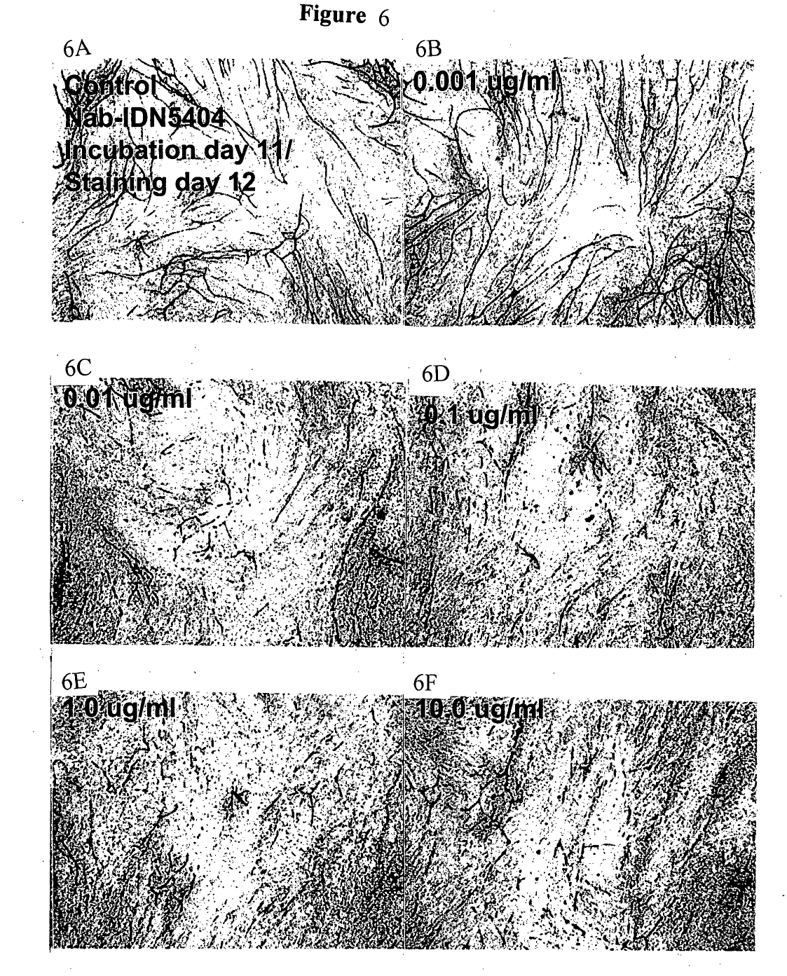

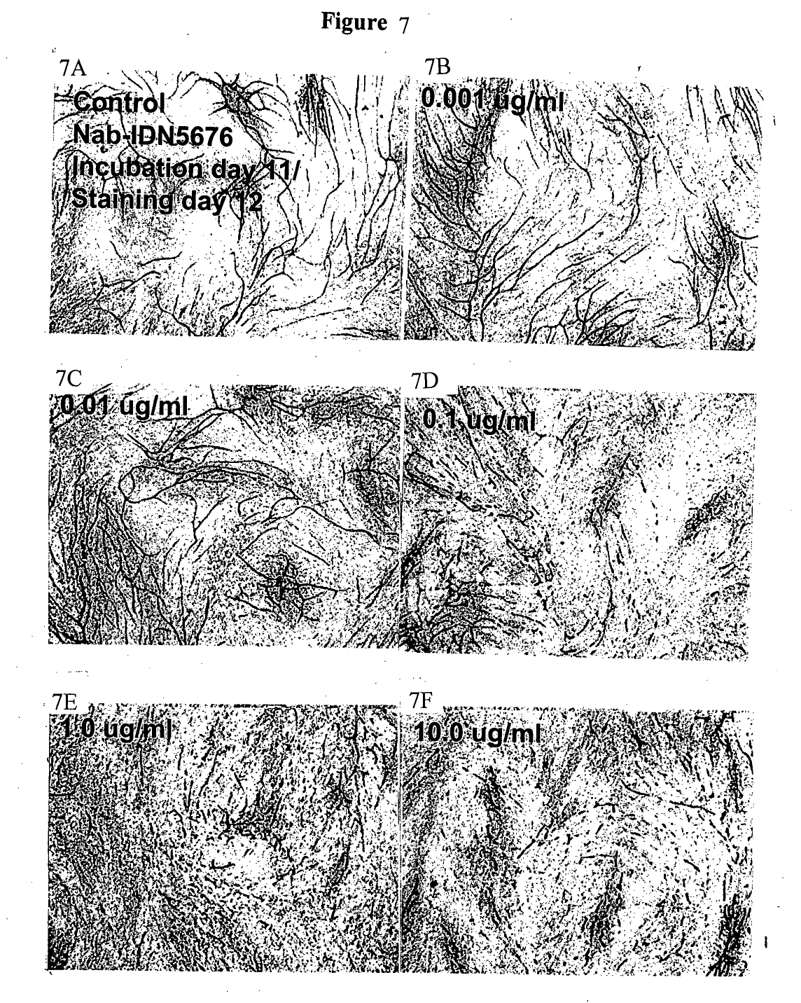

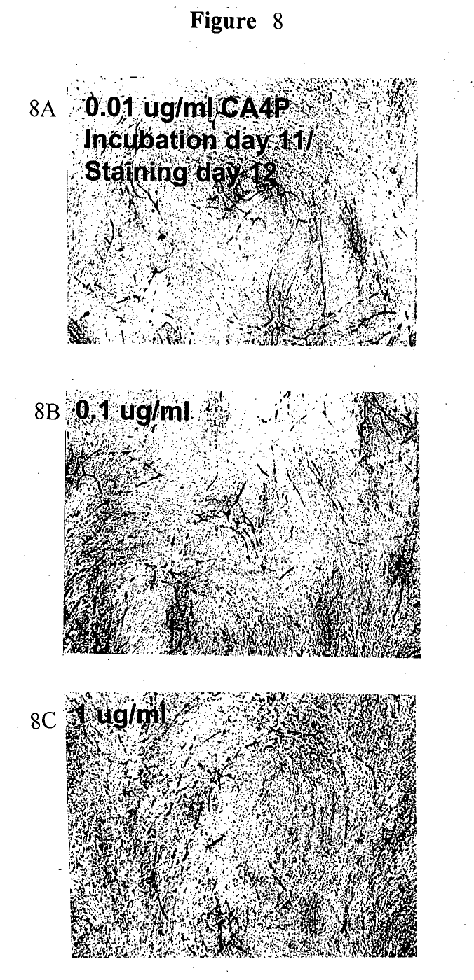

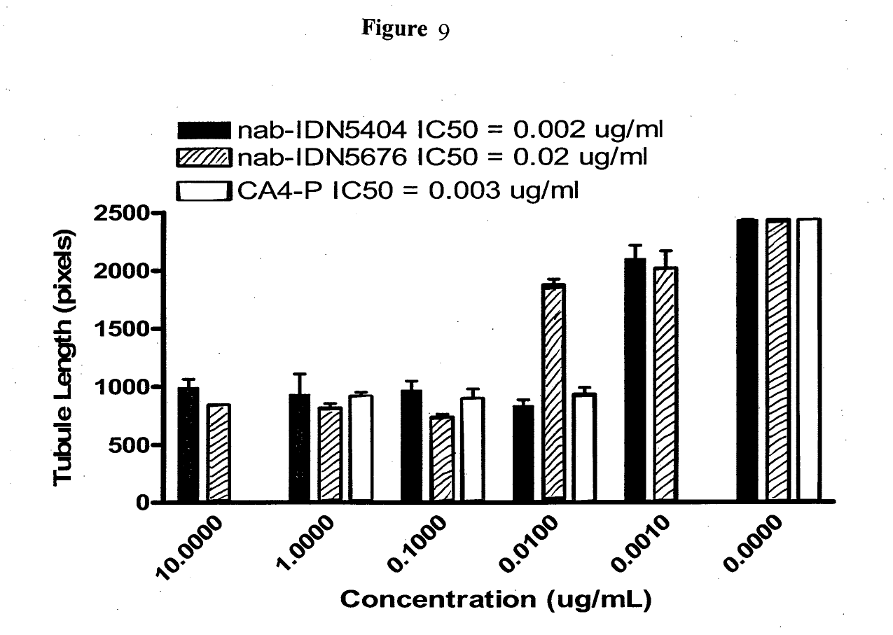

Both Nab-5404 and Nab-5676 were capable of inhibiting the formation of new microvessels (FIGS. 3 and 4). In the second experiment, both Nab-5404 and Nab-5676 were capable of inhibiting tubule formation and/or disrupting established tubules (FIG. 5). Nab-5404 was more potent than Nab-5676 in disrupting established tubules (FIGS. 6, 7, and 9). The IC50 of Nab-5404 was calculated to be 0.002 μg/ml, ten times more potent than Nab-5676 with an IC50 of 0.02 μg/ml. Nab-5404 was as effective as CA4P (IC50=0.003 μg/ml) in disrupting established tubules (FIGS. 6, 8, and 9).

### Example 4

### Anti-tumor Activity of Nab-5404 and Nab-5676

The anti-tumor activity of Nab-5404 and Nab-5676 was evaluated in a xenograft mouse model. The compounds were tested against established HT-29 colon cell tumors in vivo in two cycles, the first a low-dosage schedule followed by a second high-dosage schedule. Irinotecan was used as a positive control in the study. Mice (n=10) were separated into 8 groups with Nab-5404 and Nab-5676 mice receiving the compounds by intravenous injection. The first cycle consisted of administration of Nab-5404 or Nab-5676 every 3 days for 4 doses for days 0-14 followed by the second cycle which consisted of administration of Nab-5404 or Nab-5676 every 3 days for 4 doses for days 15-30. Irinotecan was administered by intravenous injection at a dose of 60 mg/kg every 3 days for 4 doses. The individual groups are shown in Table 2, wherein 1^{st} cycle refers to days 0-14 and 2^{nd} cycle refers to days 15-30.

TABLE 2

| | Treatment | | | |
|---|---|---|---|---|
| | 1^{st} cycle | 1^{st} cycle | 2^{nd} cycle | 2^{nd} cycle |
| Control vehicle | dose | schedule | dose | schedule |
| Irinotecan | 60 mg/kg | q3d x 4 | | |
| Nab-5404 | 3.4 mg/kg | q3d x 4 | 40 mg/kg | q3d x 4 |
| Nab-5404 | 2.5 mg/kg | q3d x 4 | 30 mg/kg | q3d x 4 |
| Nab-5404 | 1.7 mg/kg | q3d x 4 | 20 mg/kg | q3d x 4 |
| Nab-5676 | 3.4 mg/kg | q3d x 4 | 40 mg/kg | q3d x 4 |
| Nab-5676 | 2.5 mg/kg | q3d x 4 | 30 mg/kg | q3d x 4 |
| Nab-5676 | 1.7 mg/kg | q3d x 4 | 20 mg/kg | q3d x 4 |

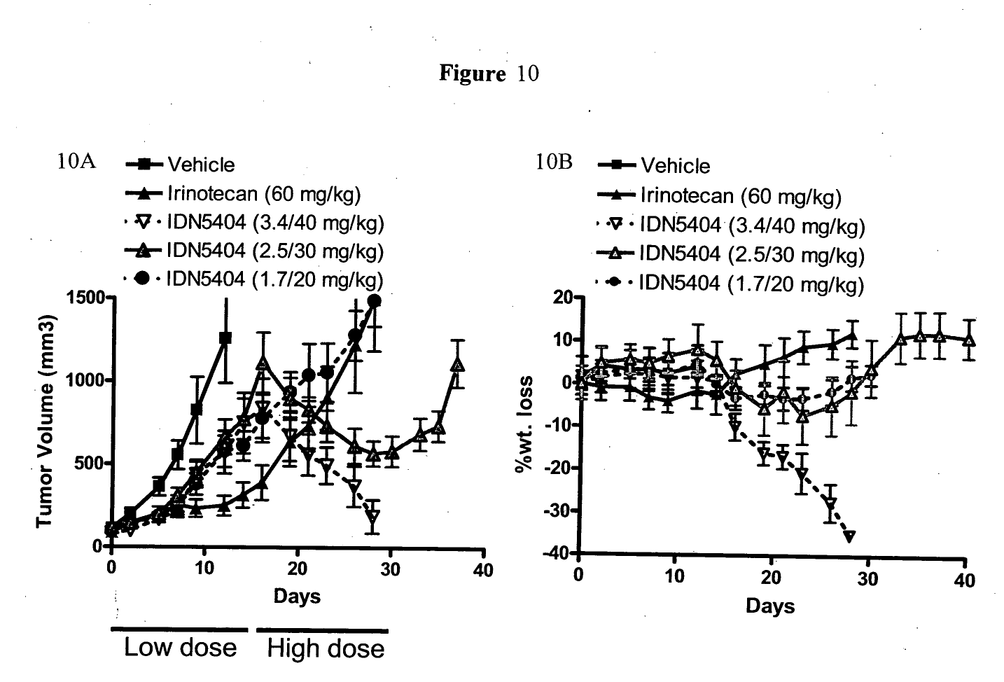

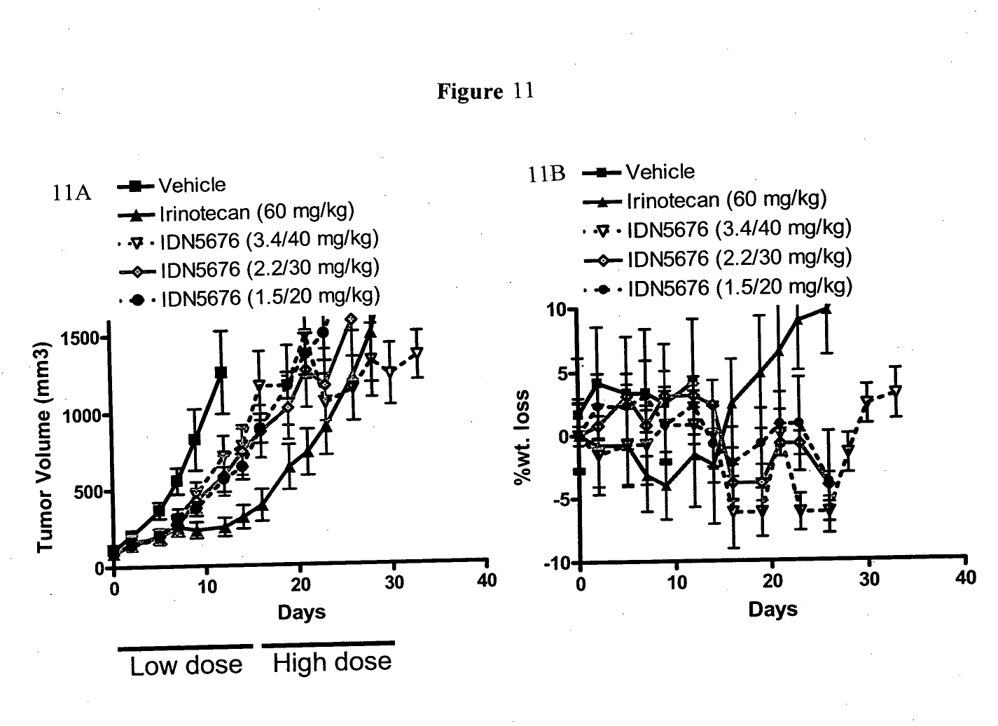

As shown in FIGS. 10 and 11, at the lower dosages, both Nab-5404 and Nab-5676 inhibited tumor growth significantly with p values of 0.02, 0.007, 0.001 for 3.4, 2.5, and 1.7 mg/kg of Nab-5404 and p values of 0.04, 0.003 and 0.0004 for 3.4, 2.5, and 1.7 mg/kg Nab-5676. The anti-tumor activities of Nab-5404 and Nab-5676 at low doses suggest that these compounds have anti-angiogenic activities. At the higher dosages, there was significantly greater tumor regression after treatment with Nab-5404 as compared to Nab-5676. Based upon weight loss data the MTD of Nab-5404 in this model was approximately 30 mg/kg with significant tumor regression.

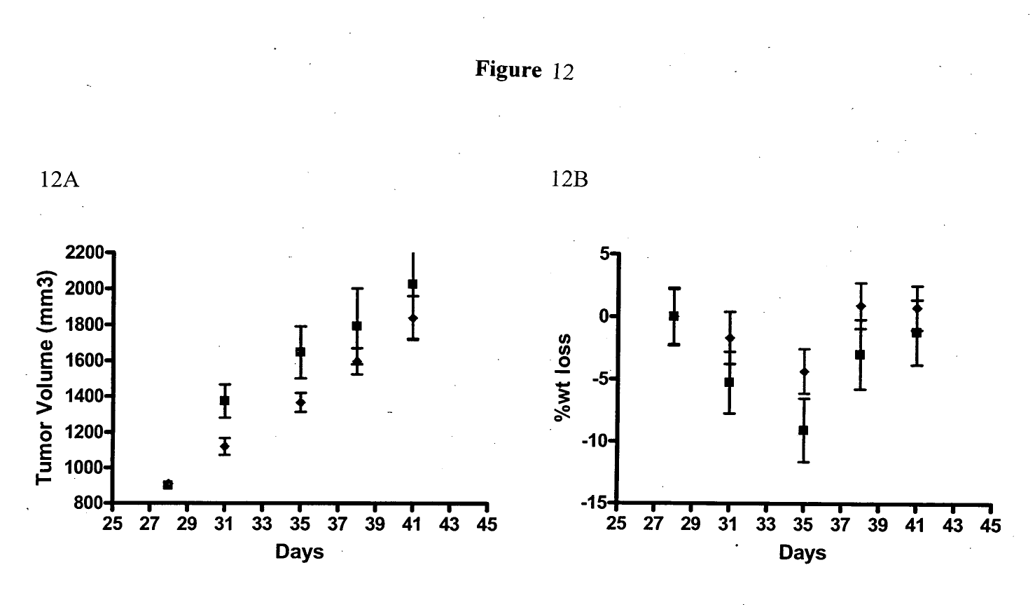

Combretastatin 4-phosphate (CA4P) was also administered to HT-29 tumor-bearing mice in a separate experiment. The mice (n=10) were treated once tumors reached 900 mm³ with either vehicle or CA4P at the reported MTD of 100 mg/kg on a schedule of every day for 4 doses. CA4P was found to be inactive in this tumor model with no difference from the control vehicle (FIG. 12).

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method of inhibiting angiogenesis in an individual, comprising administering to the individual a composition comprising a colchicine or thiocolchicine dimer, wherein the composition is in an amount that is effective in inhibiting angiogenesis, and wherein the amount of the composition is insufficient to induce significant cytotoxicity in the individual.

2. The method of claim 1, wherein the composition further comprises a carrier protein.

3. The method of claim 2, wherein the amount of the colchicine or thiocolchicine dimer in the composition is less than about 15 mg/m².

4. The method of claim 2, wherein the individual has cancer.

5. The method of claim 2, wherein the individual has a non-tumorous angiogenesis-associated disease.

6. The method of claim 2, wherein the colchicine or thiocolchicine dimer is a compound of formula (II): 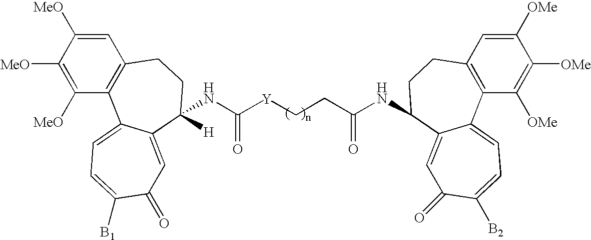
wherein:
B₁ is methoxy or methylthio group;
B₂ is a methoxy or methylthio group;
n is an integer from 0 to 8;
Y is a CH₂ group or, when n is 1, can also be a group of formula NH.

7. The method of claim 6, wherein Y is CH2.

8. The method of claim 6, wherein n is 1 and Y is NH.

9. The method of claim 6, wherein B1 and B2 are both methylthio groups.

10. The method of claim 6, wherein the compound is IDN5404 or IDN5676.

11. The method of claim 2, wherein the composition is administered at least once a week.

12. The method of claim 2, wherein the composition is administered without breaks for more than about 3 months.

13. The method of claim 2, wherein the composition is substantially free of surfactant.

14. The method of claim 2, wherein the carrier protein is albumin.

15. The method of claim 14, wherein the albumin to colchicine or thiocolchicine dimer ratio is less than about 18:1.

16. The method of claim 2, wherein the composition comprises nanoparticles comprising the carrier protein and the colchicine or thiocolchine dimer.

17. The method of claim 16, wherein the nanoparticles have an average diameter of 200 nm or less.

18. The method of claim 2, wherein the composition is Nab-5404 or Nab-5676.

19. The method claim 2, wherein the individual is a human.

20. A method of inhibiting angiogenesis in an individual having a non-tumorous angiogenesis-associated disease, comprising administering to the individual a composition comprising a colchicine or thiocolchicine dimer, wherein the composition is in an amount that is effective in inhibiting angiogenesis.

21. The method of claim 20, wherein the individual does not have cancer.

22. The method of claim 20, wherein the composition further comprises a carrier protein.

23. The method of claim 22, wherein the non-tumorous angiogenesis-associated disease is an eye disease.

24. The method of claim 23, wherein the eye disease is age-related macular degeneration, diabetic retinopathy, or neovascular glaucoma.

25. The method of claim 22, wherein the non-tumorous angiogenesis-associated disease is a cardiovascular disease.

26. The method of claim 25, wherein the cardiovascular disease is restenosis or atherosclerosis.

27. The method of claim 22, wherein the non-tumorous angiogenesis-associated disease is rheumatoid arthritis or psoriasis.

28. The method of claim 22, wherein the amount of the composition is insufficient to induce significant cytotoxicity in the individual.

29. The method of claim 22, wherein the colchicine or thiocolchicine dimer is a compound of formula (II): 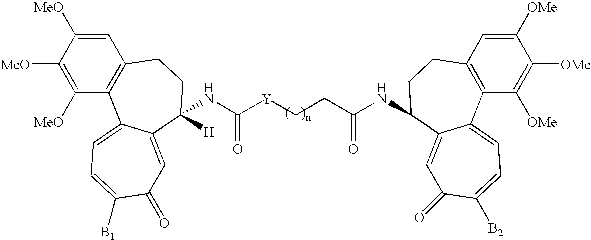
wherein:
B₁ is methoxy or methylthio group;
B₂ is a methoxy or methylthio group;
n is an integer from 0 to 8;
Y is a CH₂ group or, when n is 1, can also be a group of formula NH.

30. The method of claim 29, wherein the compound is IDN5404 or IDN5676.

31. The method of claim 22, wherein the carrier protein is albumin.

32. The method of claim 22, wherein the composition comprises nanoparticles comprising the carrier protein and the colchicine or thiocolchine dimer.

33. The method of claim 22, wherein the composition is Nab-5404 or Nab-5676.

34. The method claim 22, wherein the individual is a human.

35. A method of treating cancer in an individual, comprising administering to the individual a composition comprising a colchicine or thiocolchicine dimer, wherein the composition is in an amount that is effective in treating cancer, wherein the amount of colchicine or thiocolchicine dimer in the composition is less than about 40% of the corresponding MTD.

36. The method of claim 35, wherein the composition further comprises a carrier protein.

37. The method of claim 35, wherein the amount of the colchicine or thiocolchicine dimer in the composition is less than about 15% of the corresponding MTD.

38. The method of claim 36, wherein the cancer is a solid tumor.

39. The method of claim 38, wherein the cancer is a metastatic solid tumor.

40. The method of claim 36, wherein the colchicine or thiocolchicine dimer is a compound of formula (II): 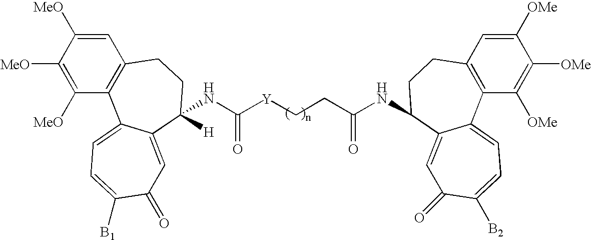
wherein:
B₁ is methoxy or methylthio group;
B₂ is a methoxy or methylthio group;
n is an integer from 0 to 8;
Y is a CH₂ group or, when n is 1, can also be a group of formula NH.

41. The method of claim 40, wherein the compound is IDN5404 or IDN5676.

42. The method of claim 36, wherein the carrier protein is albumin.

43. The method of claim 36, wherein the composition comprises nanoparticles comprising the carrier protein and the colchicine or thiocolchine dimer.

44. The method of claim 36, wherein the composition is Nab-5404 or Nab-5676.

45. The method claim 36, wherein the individual is a human.

46. A method of treating cancer in an individual, comprising administering to the individual a composition comprising a colchicine or thiocolchicine dimer, wherein the composition is in an amount that is effective in treating cancer, and wherein the amount of the composition is insufficient to induce significant cytotoxicity in the individual.

47. The method of claim 45, wherein the composition further comprises a carrier protein.

48. A unit dosage form comprising a composition comprising colchicine or thiocolchicine dimer in an amount that is effective in inhibiting angiogenesis, wherein the amount of the composition is insufficient to induce significant cytotoxicity in the individual.

49. The unit dosage form of claim 48, wherein the unit dosage form further comprises a carrier protein.

50. The unit dosage form of claim 49, wherein the unit dosage form comprises a colchicine or thiocolchicine dimer in an amount that is sufficient to deliver about 0.25 mg/m² to about 100 mg/m² colchicine or thiocolchicine dimer to a subject.

51. The unit dosage form of claim 49, wherein the unit dosage form comprises about 0.05 mg to about 200 mg colchicine or thiocolchicine dimer.

52. The unit dosage form of claim 49, wherein the colchicine or thiocolchicine dimer is a compound of formula (II): 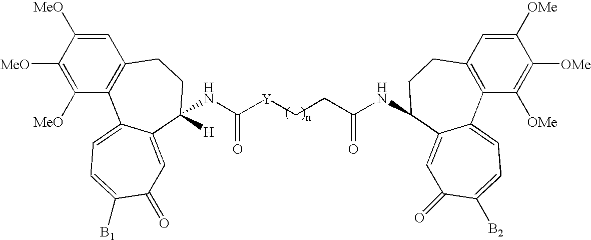
wherein:
B₁ is methoxy or methylthio group;
B₂ is a methoxy or methylthio group;
n is an integer from 0 to 8;
Y is a CH₂ group or, when n is 1, can also be a group of formula NH.

53. The unit dosage form of claim 52, wherein the compound is IDN5404 or IDN5676.

54. The method of claim 49, wherein the carrier protein is albumin.

55. The unit dosage form of claim 49, wherein the composition comprises nanoparticles comprising the carrier protein and the colchicine or thiocolchine dimer.

56. The unit dosage form of claim 55, wherein the composition is Nab-5404 or Nab-5676.

57. A pharmaceutical composition comprising a colchicine or thiocolchicine dimer, a carrier protein, and a pharmaceutically acceptable carrier suitable for delivery to the eye or intraarterial injection.

58. The pharmaceutical composition of claim 57, wherein the pharmaceutically acceptable carrier is suitable for intraocular injection.

59. The pharmaceutical composition of claim 57, wherein the pharmaceutically acceptable carrier is suitable for topical application to the eye.

60. The pharmaceutical composition of claim 57, wherein the carrier protein is albumin.

61. The pharmaceutical composition of claim 57, wherein the composition comprises nanoparticles comprising the carrier protein and the colchicine or thiocolchine dimer.
